(19) United States
(12) Patent Application Publication
(10) Pub. No.: US 2024/0252102 A1
(43) Pub. Date: Aug. 1, 2024

(54) SYSTEMS AND METHODS OF USING NANOMEMBRANE ELECTRONICS

(71) Applicants: Woon-Hong YEO, Atlanta, GA (US); Hyojung J. CHOO, Atlanta, GA (US); GEORGIA TECH RESEARCH CORPORATION, Atlanta, GA (US); EMORY UNIVERSITY, Atlanta, GA (US)

(72) Inventors: Woon-Hong YEO, Atlanta, GA (US); Hyojung J. CHOO, Atlanta, GA (US)

(21) Appl. No.: 18/564,391

(22) PCT Filed: May 27, 2022
(86) PCT No.: PCT/US2022/031339

§ 371 Date: Nov. 27, 2023

Related U.S. Application Data
(60) Provisional application No. 63/194,113, filed on May 27, 2021.

(51) Int. Cl. A61B 5/00; A61B 5/257; A61B 5/28; A61B 5/291; A61B 5/296

(52) U.S. Cl. CPC A61B 5/4519; A61B 5/257; A61B 5/28; A61B 5/291; A61B 5/296; A61B 5/4842; A61B 5/4848; A61B 5/6832; A61B 2503/40; A61B 2503/42; A61B 2560/0468; A61B 2562/0219; A61B 2562/028; A61B 2562/125; A61B 2562/164; A61B 2562/166

(57) ABSTRACT

Described herein are wireless nanomembrane non-invasive system that integrates skin-wearable printed sensors and electronics and methods that can be used to monitor an electrophysiological parameter of a subject or to identify a therapeutic agent. The systems can include wearable devices, including skin-wearable printed sensors; and electronics for real-time, continuous monitoring of electrophysiological parameters of a subject.

### CROSS-REFERENCE TO RELATED APPLICATIONS

The application claims the benefit of U.S. Provisional Application No. 63/194,113, filed May 27, 2021, which is hereby incorporated herein by reference in its entirety.

### STATEMENT REGARDING FEDERALLY SPONSORED RESEARCH OR DEVELOPMENT

This invention was made with government support under grant no. R01AR071397, awarded by the National Institute of Health. The government has certain rights in the invention.

### BACKGROUND

Animal models can provide invaluable information in the pursuit of scientific and medical knowledge and the development of new drugs and treatments, particularly in the preclinical investigations of such research and development. An animal model is a non-human species used in biomedical and drug research because it can mimic aspects of a biological process or disease found in humans. Animal models (e.g., mice, rats, zebrafish, and others) share similarities to humans in their anatomy, physiology, or response to a pathogen that allows researchers to extrapolate the results of animal model studies to better understand human physiology and disease. By using animal models, researchers can perform experiments that would be impractical or ethically prohibited with humans.

In most pre-clinical studies, animal models are introduced, or periodically introduced, with an agent of interest or a treatment. The animal model would then be euthanized following a pre-defined time period to evaluate the physiological response of the animal model.

There is a benefit to extracting other clinically relevant information from animal models in a non-invasive manner.

### SUMMARY

Described herein are wireless nanomembrane non-invasive systems that integrate skin-wearable printed sensors and electronics and methods that can be used to monitor an electrophysiological parameter of a subject or to identify a therapeutic agent. The systems can include wearable devices, including skin-wearable printed sensors; and electronics for real-time, continuous monitoring of electrophysiological parameters of a subject.

The methods described can monitor an electrophysiological parameter of a subject. The method can include acquiring signals from a wearable device described herein; and assessing disease progression on the subject, an injury on the subject, or any combination thereof using the acquired signals to provide real-time, continuous monitoring of the electrophysiological parameters of the subject.

The methods described can identify a therapeutic agent; the method can include: contacting a wearable device comprising a skin-wearable printed sensor with a subject's skin; acquiring signals from the wearable device on the subject's skin; administering an agent of interest to the subject; acquiring signals from the wearable device on the subject's skin following administration of the agent of interest; comparing the signals of the subject before and after administration of the agent of interest; and analyze a result from the comparison step to assess a physiological parameter of the subject; wherein the physiological parameter provides an indication that the agent of interest is a therapeutic agent.

The methods described can identify a diagnostic agent; the method can include: contacting a wearable device comprising a skin-wearable printed sensor with a subject's skin; acquiring signals from the wearable device on the subject's skin; administering an agent of interest to the subject; acquiring signals from the wearable device on the subject's skin following administration of the agent of interest; comparing the signals of the subject before and after administration of the agent of interest; and analyze a result from the comparison step to assess a physiological parameter of the subject; wherein the physiological parameter provides an indication that the agent of interest is a diagnostic agent. In some embodiments, the wearable device can include a system described herein.

In some embodiments, the subject can be an animal model also described herein. The animal models can include an animal subject, including the wearable device described herein over the animal subject's skin, wherein the animal subject was administered an agent of interest, was subjected to an injury, or any combination thereof. In some embodiments, the animal subject was administered an agent of interest. In some embodiments, the animal subject was subjected to an injury. In some embodiments, the animal subject was administered an agent of interest and was subjected to an injury.

In some embodiments, the injury can include an induced masseter muscle injury. In some embodiments, the animal model can be a craniofacial VML model. In some embodiments, the craniofacial VML model can include an animal subject having been subjected to biopsy punch-induced masseter muscle injury. In some embodiments, the animal model exhibits impaired muscle regeneration and imbalanced muscle resident stem cell activities.

### DESCRIPTION OF DRAWINGS

FIG. 1 shows an example system configured for real-time, continuous monitoring of an animal model in accordance with an illustrative embodiment.

FIGS. 2A and 2B collectively show an example method of operating a wearable sensor system to assess an agent of interest or treatment by monitoring the disease, injury, or condition progression of a subject (e.g., to identify or confirm a therapeutic agent or its efficacious dosage) in accordance with an illustrative embodiment.

FIGS. 3A, 3B, 3C, and 3D each shows an example skin-wearable sensor system of FIG. 1 in accordance with an illustrative embodiment.

FIG. 4 shows an example circuit and layout of the sensor system in accordance with an illustrative embodiment.

FIG. 5 shows a printing process for graphene electrodes and a microfabrication process for a thin film-based circuit, e.g., for the sensor system of FIG. 1, in accordance with an illustrative embodiment.

FIGS. 6A-6E show an overview of a study to evaluate real-time functional assay of craniofacial VML in mice using wireless nanomembrane electronics.

FIGS. 7A-7H show results of characterizations of graphene membrane electrodes and wireless soft circuits in the study of FIG. 6.

FIGS. 8A-8F show a study to establish craniofacial VML and defective masseter muscle regeneration.

FIGS. 9A-9G show the operation of a wireless, wearable EMG system to assess the functionality of VML-injured masseter muscles.

FIGS. 10A-10G show operation for monitoring the functional recovery of VML-injured masseter muscles post-transplantation.

FIGS. 11A-11B show results of computational mechanical simulations.

FIGS. 12A-12D show results of filtered EMG signals of a post-VML-injured mouse.

FIGS. 13A-13E show results of dysregulated stem cells in masseter muscles three days post-VML injury.

FIG. 14 shows filtered EMG signals of an uninjured mouse while eating.

### DETAILED DESCRIPTION

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference.

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entireties and to the same extent as if each reference was individually incorporated by reference.

### Definitions

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include the one particular value and/or the other particular value.

By “comprising” or “containing” or “including,” is meant that at least the name compound, element, particle, or method step is present in the composition or article or method but does not exclude the presence of other compounds, materials, particles, method steps, even if the other such compounds, material, particles, method steps have the same function as what is named.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

It is understood that when combinations, subsets, groups, etc. of elements are disclosed (e.g., combinations of components in a composition or combinations of steps in a method), specific reference of each of the various individual and collective combinations and permutations of these elements may not be explicitly disclosed, each is specifically contemplated and described herein.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

As discussed herein, a “subject” may be any applicable animal, or other organisms, living or dead, or other biological or molecular structure or chemical environment, and may relate to particular components of the subject, for instance, specific tissues or fluids of a subject (e.g., animal tissue in a particular area of the body of a living subject), which may be in a particular location of the subject, referred to herein as an “area of interest” or a “region of interest.”

It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to humans (e.g., rat, dog, pig, monkey), etc.

“Administration” to a subject includes any route of introducing or delivering to a subject an agent. Administration can be carried out by any suitable route, including oral, topical, transcutaneous, transdermal, intra-joint, intra-arteriole, intradermal, intraventricular, intralesional, intranasal, rectal, vaginal, by inhalation, via an implanted reservoir, parenteral (e.g., subcutaneous, intravenous, intramuscular, intra-articular, intra-synovial, intracisternal, intrathecal, intraperitoneal, intrahepatic, intralesional, and intracranial injections or infusion techniques), and the like. “Concurrent administration,” “administration in combination,” “simultaneous administration,” or “administered simultaneously,” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route that introduces or delivers the agent to extensive areas of the subject's body (e.g., greater than 50% of the body), for example, through the entrance into the circulatory or lymph systems. By contrast, “local administration” refers to the introducing or delivery to a subject an agent via a route that introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

As used here, the “agent of interest” may be therapeutic, diagnostic, or prophylactic agents. The agent may be an organic molecule (e.g., a therapeutic agent, a drug), inorganic molecule, nucleic acid, protein, amino acid, peptide, polypeptide, polynucleotide, targeting agent, isotopically labeled organic or inorganic molecule, vaccine, immunological agent, etc. In some embodiments, the term “agent of interest” is used herein to refer to a chemical compound or composition that may have a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc. In some embodiments, the agent of interest can be an agent determined to be a suitable therapeutic agent for improving an injury.

As used here, the terms “beneficial agent” and “active agent” are used interchangeably herein to refer to a chemical compound or composition that has a beneficial biological effect. Beneficial biological effects include both therapeutic effects, i.e., treatment of a disorder or other undesirable physiological condition, and prophylactic effects, i.e., prevention of a disorder or other undesirable physiological condition. The terms also encompass pharmaceutically acceptable, pharmacologically active derivatives of beneficial agents specifically mentioned herein, including, but not limited to, salts, esters, amides, prodrugs, active metabolites, isomers, fragments, analogs, and the like. When the terms “beneficial agent” or “active agent” are used, then, or when a particular agent is specifically identified, it is to be understood that the term includes the agent per se as well as pharmaceutically acceptable, pharmacologically active salts, esters, amides, prodrugs, conjugates, active metabolites, isomers, fragments, analogs, etc.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, or composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease activity, response, condition, disease, or other biological parameters. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

“Inactivate,” “inactivating,” and “inactivation” means to decrease or eliminate an activity, response, condition, disease, or other biological parameters due to a chemical (covalent bond formation) between the ligand and its biological target.

By “reduce” or other forms of the word, such as “reducing” or “reduction,” is meant lowering of an event or characteristic (e.g., tumor growth). It is understood that this is typically in relation to some standard or expected value; in other words, it is relative, but it is not always necessary for the standard or relative value to be referred to. For example, “reduces tumor growth” means reducing the rate of growth of a tumor relative to a standard or a control.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to a reduction in severity and/or frequency of symptoms, elimination of symptoms and/or underlying cause, prevention of the occurrence of symptoms and/or their underlying cause, and improvement or remediation of damage.

By “prevent” or other forms of the word, such as “preventing” or “prevention,” is meant to stop a particular event or characteristic, to stabilize or delay the development or progression of a particular event or characteristic, or minimize the chances that a particular event or characteristic will occur. Prevent does not require comparison to control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used unless specifically indicated otherwise, the use of the other word is also expressly disclosed. For example, the terms “prevent” or “suppress” can refer to a treatment that forestalls or slows the onset of a disease or condition or reduces the severity of the disease or condition. Thus, if a treatment can treat a disease in a subject having symptoms of the disease, it can also prevent or suppress that disease in a subject which has yet to suffer some or all of the symptoms. As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts.

An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

As used herein, a “therapeutically effective amount” of a therapeutic agent refers to an amount that is effective in achieving a desired therapeutic result, and a “prophylactically effective amount” of a therapeutic agent refers to an amount that is effective in preventing an unwanted physiological condition. Therapeutically effective and prophylactically effective amounts of a given therapeutic agent will typically vary with respect to factors such as the type and severity of the disorder or disease being treated and the age, gender, and weight of the subject. The term “therapeutically effective amount” can also refer to an amount of a therapeutic agent or a rate of delivery of a therapeutic agent (e.g., amount over time), effective in facilitating a desired therapeutic effect. The precise desired therapeutic effect will vary according to the condition to be treated, the tolerance of the subject, the drug and/or drug formulation to be administered (e.g., the potency of the therapeutic agent (drug), the concentration of drug in the formulation, and the like), and a variety of other factors that are appreciated by those of ordinary skill in the art.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included in the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate-buffered saline solution, water, emulsions (such as an oil/water or water/oil emulsion), and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

Also, as used herein, the term “pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

A “control” is an alternative subject or sample used in an experiment for comparison purposes. A control can be “positive” or “negative.”

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

### Example System

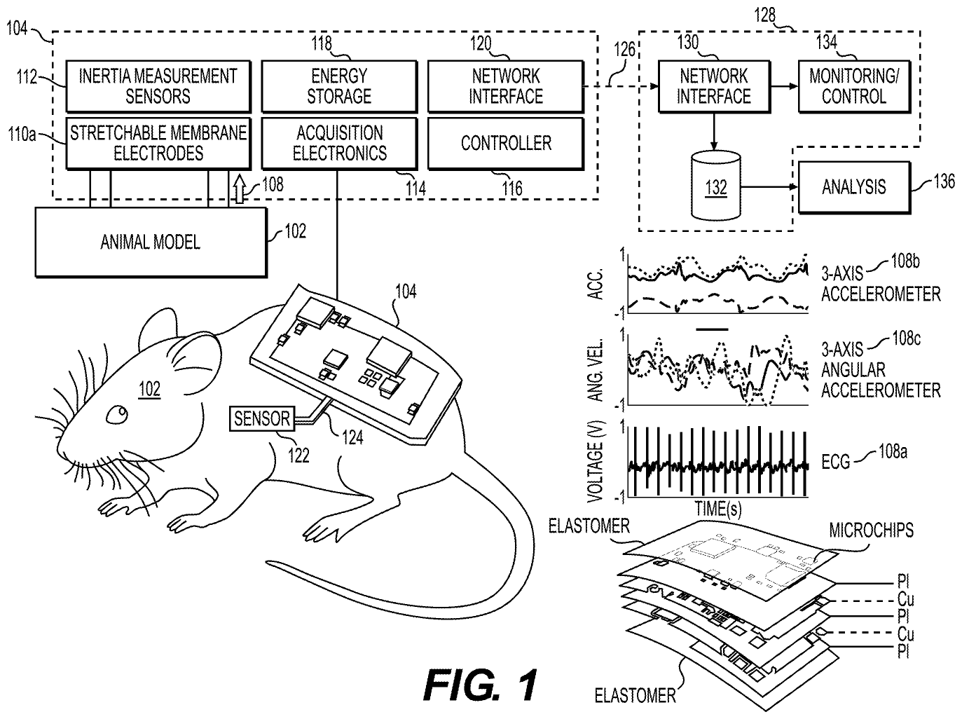

FIG. 1 shows an example system 100 configured for real-time, continuous monitoring of an animal model in accordance with an illustrative embodiment. The system 100 can be used to monitor physiological parameters or metrics of the animal model to allow for the assessment on an ongoing basis of an agent of interest or surgical treatment. The system 100 can augment or replace current experimental evaluation paradigms and procedures for the assessment of therapeutic agents or surgically induced procedures via animal models. The system 100 is miniaturized to integrate with animal models for portable real-time monitoring over an extended period of time.

In the example shown in FIG. 1A, the system 100 includes an animal model 102 that is coupled to or laminated with a wireless, non-invasive skin-wearable sensor system 104. The sensor system 104 includes a set of one or more sensors 106 (shown as 106a) to acquire one or more biophysical signals 108 from the animal model 102. The sensors 106 can acquire time-series data or channels of them, such as inertia, acceleration, orientation, temperature, or sound. The acquired time-series data of sensor 106 may also include electromyogram (EMG), electrocardiogram (ECG), electroencephalogram (EEG), phonocardiogram, voltage potential, impedance, and acoustics. The sensors 106 may be configured as a sensor array to acquire time-series array data, e.g., time-series data or images.

In the example shown in FIG. 1, the skin-wearable sensor system 104 includes a main sensor set comprising a stretchable sensor 110, e.g., configured as a stretchable membrane electrode 110a, and auxiliary sensor set 112, e.g., comprising inertia measurement sensors 112, as well acquisition electronics 114, controller 116, energy storage module 118, and network interface 120. The stretchable membrane sensor 110, or sensor assembly, may be mounted on the underside of the skin-wearable sensor system 104, e.g., through a software membrane underlayer. In some embodiments, the stretchable membrane sensor 110 may be configured as an external sensor (shown as 122 in FIG. 1) that can be mounted on another region of the animal model 102 and connected to the sensor system 104 through an interconnect 124. The stretchable membrane sensor 110, or sensor assembly, may be configured with a stretchable electrode to directly acquire the biophysical signals 108 from the animal model 102 or with a stretchable contact pad that can couple to an integrated sensor circuit.

The exemplary system 100 may include ultrathin, low-profile, lightweight, and stretchable membrane sensors based on biocompatible thin-film soft circuits (e.g., graphene) for data processing to provide seamless mounting on the skin of alive and moving animal models (e.g., mice or rats) without disrupting their natural behavior. In addition, the compact device integration on a soft elastomeric platform can provide comfortable wearability without motion artifacts caused by cumbersome wires and rigid system. The use of non-invasive and ergonomic monitoring system/device allows movement during measurement, thus allowing the system to monitor the physiological response in a natural ambulatory environment.

The skin-wearable sensor system 104 is configured to communicate through a short-range communication channel 126 to a data acquisition system 128 configured with a network interface 130, data storage 132, and monitoring and control module 134 (also referred to as a controller 134). The acquired data can be subsequently analyzed in an analysis system or operation 136. The data acquisition system 128 can be a customized data storage system, a standard computing device configured with internal data acquisition hardware, or a standard computing device configured with external data acquisition hardware.

As noted, the skin-wearable sensor system 104 includes auxiliary sensors, such as inertia measurement sensors 112, that can provide inertia, acceleration, and orientation information relating to movement or activity. The inertia signals (e.g., inertia, acceleration, orientation information) of the inertia measurement sensors 112 can be used to remove noise or movement artifacts from the stretchable sensor 110.

The acquisition electronics 128 may include analog-to-digital convertors or capacitance-to-digital converters, trans-impedance amplifiers or other amplifier circuitries, appropriate filters (e.g., low pass and/or high pass filters), and corresponding circuitries for voltage regulations and clocks.

The controller 134 includes a processing unit, which may be a standard programmable processor that performs arithmetic and logic operations necessary for the operation of the computing device. As used herein, processing unit and processor refers to a physical hardware device that executes encoded instructions for performing functions on inputs and creating outputs, including, for example, but not limited to, microprocessors (MCUs), microcontrollers, graphical processing units (GPUs), and application-specific circuits (ASICs). Thus, while instructions may be discussed as executed by a processor, the instructions may be executed simultaneously, serially, or otherwise executed by one or multiple processors. The computing device may also include a bus or other communication mechanism for communicating information among various components of the computing device. Multiple processors may be employed by controller 134.

The data acquisition system 128 can be a customized data storage system, a standard computing device configured with internal data acquisition hardware, or a standard computing device configured with external data acquisition hardware. The data acquisition system 128 may include an interface and display to present one or more biophysical signals 108 from the animal models. The data acquisition system 128 can acquire and present data and log data from multiple skin-wearable sensor systems 104 as well as from other instrumentations. In some embodiments, the monitoring and control module 134 is configured with a web service module that can be curated or present one or more biophysical signals 108 through a web portal hosted by the web service module. In some embodiments, the monitoring and control module 134 is configured to interface with a cloud infrastructure to provide the acquired biophysical and instrumentation data to a cloud-based analysis system or to remove servers through a cloud-based storage infrastructure.

Examples of biophysical signals 108 are shown in FIG. 1 as an electrocardiogram 108a acquired via stretchable membrane sensor 110 and accelerometer and angular acceleration measurements 108b, 108c acquired via the inertia measurement sensors 112.

The network interface 130 is configured to communicate between the skin-wearable sensor system 104 and the data acquisition system 128. The network interfaces (e.g., 120, 130) may include a low power chipset for a universal serial bus (USB) interface, serial interfaces, wireless local area network (WLAN), or radio transceivers such as Bluetooth, wireless USB, or other short-range communication protocols.

The energy storage module 118 is configured to provide energy for the skin-wearable sensor system 104 and sensors. The energy storage module 118 may include a rechargeable circuit and rechargeable batteries (e.g., Lithium or nickel-cadmium) that can be wirelessly recharged via inductive charging operations. In some embodiments, the energy storage module 118 includes a power converter and connects to a power source through a wire connection.

The data storage 132 may be a local data store of a computing device. In some embodiments, the data storage 132 is a cloud-based data store. A data store is a repository for persistently storing and managing collections of data. In some embodiments, the data store may maintain the files in a hierarchical database.

The analysis system or operation 136 may include statistical analysis of the acquired biophysical signals 108. In some embodiments, the analysis system or operation 136 is configured to clean and filter the signals 108, e.g., to remove motion artifacts. In some embodiments, the statistical analysis may include machine learning-based analysis.

The analysis system, in some embodiments, is configured to generate models from ML features and employ the ML features in a supervised or unsupervised machine learning operation to generate an estimated value (e.g., score) for the likelihood of effect of the agent of interest or treatment. In addition to the machine learning features described above, the analysis system can be implemented using one or more artificial intelligence and machine learning operations. The term “artificial intelligence” can include any technique that enables one or more computing devices or comping systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include but are not limited to autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the model discovers a pattern (e.g., structure, distribution, etc.) within an unlabeled or labeled data set. In a semi-supervised model, the model learns a function that maps an input (also known as feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers such as input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., error such as L1 or L2 loss), during training. This disclosure contemplates that any algorithm that finds the minimum of the cost function can be used. LR classifiers are known in the art and are therefore not described in further detail herein.

### Example Method

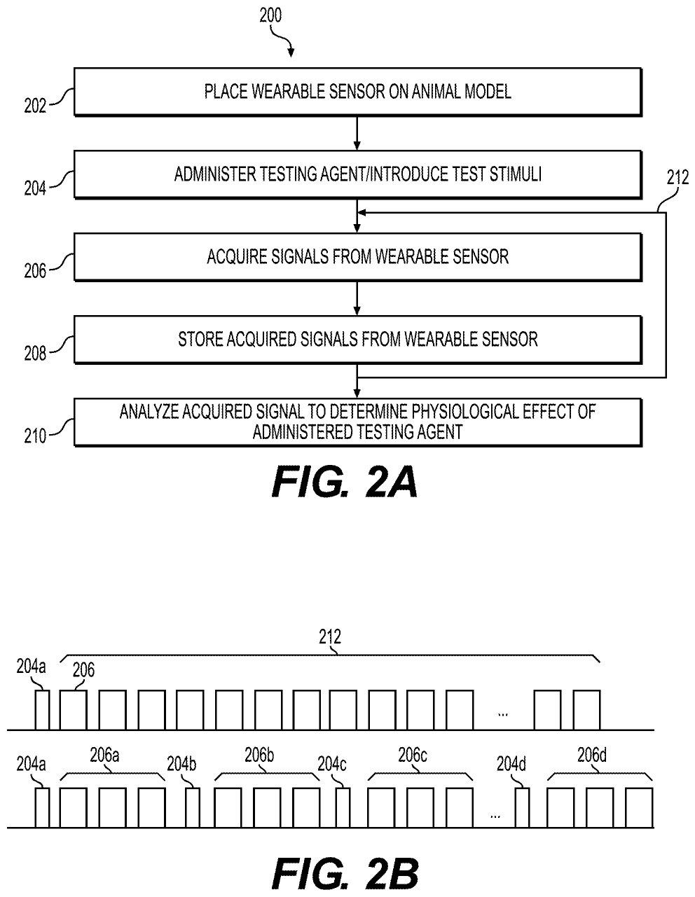

FIGS. 2A and 2B collectively show an example method 200 of operating a wearable sensor system 104 to assess an agent of interest or treatment by monitoring disease, injury, or condition progression of a subject (e.g., to identify or confirm a therapeutic agent or its efficacious dosage) in accordance with an illustrative embodiment. Method 200 includes placing (202) a wearable device (e.g., 104) comprising a wearable printed sensor (e.g., 110) on an animal model (e.g., 102), e.g., over the skin. Method 200 then includes administering (204) a testing agent or introducing test stimuli to the animal model (e.g., 102).

In some embodiments, the therapeutic agent can improve an injury on the subject.

In some embodiments, the subject was administered an agent of interest. In some embodiments, the subject was subjected to an injury. In some embodiments, the subject was administered an agent of interest and was subjected to an injury.

It is understood that the term “injury” refers to a damage to the body of a subject caused by an external force. An injury can include, but is not limited to, a wound, head injury, penetrating head injury, closed head injury, muscle injury, masseter muscle injury brain injury, acquired brain injury, coup contrecoup injury, diffuse axonal injury, frontal lobe injury, nerve injury, spinal cord injury, brachial plexus injury, sciatic nerve injury, injury of axillary nerve, soft tissue injury, tracheobronchial injury, acute kidney injury, anterior cruciate ligament injury, musculoskeletal injury, articular cartilage injuries, acute lung injury, pancreatic injury, thoracic aorta injury, biliary injury, Lisfranc injury, knee injury, medial knee injury, back injury, hand injury, chest injury. In some embodiments, the injury can include an induced masseter muscle injury.

A wound is an injury in which skin is torn, cut, or punctured (an open wound) or where blunt force trauma causes a contusion (a closed wound). A wound can be defined as any damaged region of tissue where fluid may or may not be produced. In addition, a wound or ulceration can be produced by traumatic or pathogenic disruption of an epithelial layer, such as the gastrointestinal, renal, urethral, or ureteral epithelium; or by disruption of an endothelial layer, such as the vascular or cardiac endothelium. Examples of such wounds include, but are not limited to, abdominal wounds or other large or incisional wounds, either as a result of surgery, trauma, sternotomies, fasciotomies, or other conditions, dehisced wounds, acute wounds, chronic wounds, subacute and dehisced wounds, traumatic wounds, vascular wounds (e.g., a venous ulcer, arterial ulcer), flaps and skin grafts, surgical wounds, lacerations, abrasions, contusions, hematomas, burns, diabetic ulcers, pressure ulcers, stoma, cosmetic wounds, trauma ulcers, neuropathic ulcers, a venous ulcer, arterial ulcers, chronic wound, non-healing wounds, or any combination thereof. Wounds may include readily accessible and difficult to access wounds, exposed and concealed wounds, large and small wounds, regular and irregular shaped wounds, and planar and topographically irregular, uneven, or complex wounds. The wound can be present on a site selected from the torso, limb, and extremities such as heel, sacrum, axial, inguinal, shoulder, neck, leg, foot, digit, knee, axilla, arm, and forearm, elbow, hand or any combination thereof. In some embodiments, the wound can be a vascular wound. In some embodiments, the wound can be a surgical wound. In some embodiments, the wound can be a venous ulcer. In some embodiments, the wound can be an arterial ulcer. In some embodiments, the wound can be present on a limb and extremities. In some embodiments, the wound can be a non-healing wound. Non-healing wounds refer to wounds that fail to progress in a timely manner, usually within a timeframe of 4 weeks to 3 months (e.g., from 1 month to 2 months, from 2 months to 3 months, or from 1.5 months to 2.5 months). In some embodiments, the wound can exhibit delayed healing. For example, the wound fails to progress in a timely manner, usually within a timeframe of 4 weeks to 3 months (e.g., from 1 month to 2 months, from 2 months to 3 months, or from 1.5 months to 2.5 months).

In some embodiments, the method can be a method of monitoring volumetric muscle loss (VML); the method can include acquiring EMG signals from a wearable device placed over a target muscle of a subject; and assessing VML-injured masseter muscles using the acquired EMG signals to provide real-time, continuous monitoring of VML.

Method 200 then includes acquiring (206) biophysical signals (e.g., 108) on an ongoing basis (212) from the wearable device (e.g., 104) comprising a skin-wearable printed sensor over a subject's skin. Method 200 includes storing (208) the biophysical signals (e.g., via the data acquisition system 128).

Method 200 then includes analyzing (210) the acquired signals by assessing disease progression on the subject, an injury on the subject, or any combination thereof using the acquired signals to provide real-time, continuous monitoring of the electrophysiological parameter of a subject.

In some embodiments, the administration (204) of the testing agent or test stimuli to the animal model (e.g., 102) may be performed once or may be performed multiple times over the course of a test. In an example of FIG. 2B, the administration 204 (shown as 204a) of the testing agent or test stimuli to the animal model (e.g., 102) is shown to be performed once. The wearable device (e.g., 104), e.g., a skin-wearable printed sensor, can then acquire the biophysical signals (e.g., 108) on an ongoing basis (212). In some embodiments, the period of data acquisition is over a week or a month.

In another example of FIG. 2B, the administration 204 (shown as 204a, 204b, 204c, 204d) of the testing agent or test stimuli to the animal model (e.g., 102) is shown to be performed multiple times over the course of a test. The wearable device (e.g., 104) can acquire (206) the biophysical signals (e.g., 108) on an ongoing basis (212). In some embodiments, the period of data acquisition is over a week or a month.

The skin-wearable printed sensor may include one or more stretchable graphene sensors, e.g., configured as part of an electrical sensor, an impedance sensor, an infrared sensor, or any combination thereof, e.g., to acquire an electrocardiogram (ECG) sensor, an electroencephalogram (EEC) sensor, an electromyogram (EMG) sensor, or any combination thereof. The skin-wearable printed sensor may include at least two electrodes, a conductive flexible film, and an elastomeric substrate. In some embodiments, the polymer layer can include polyimide (PI).

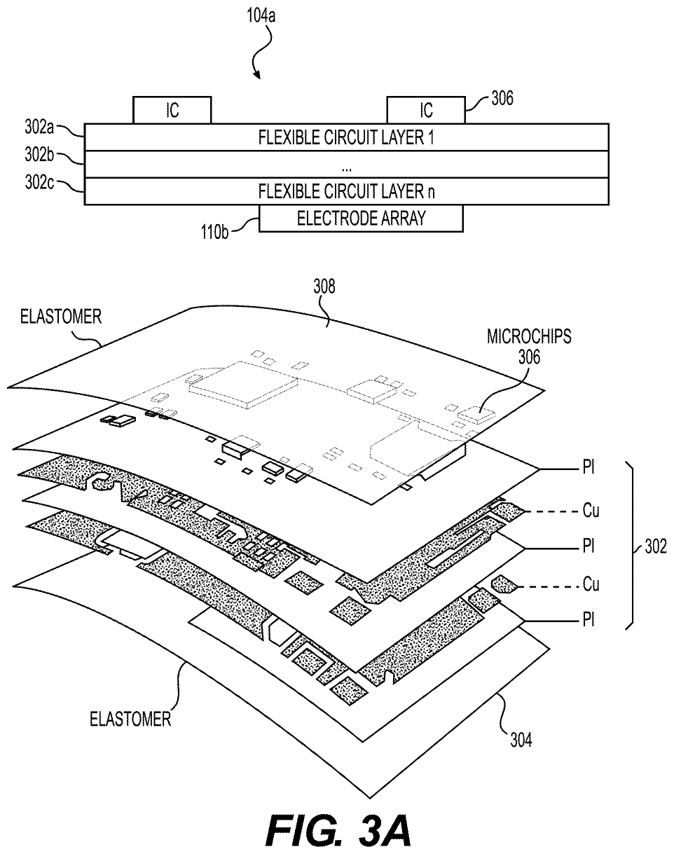

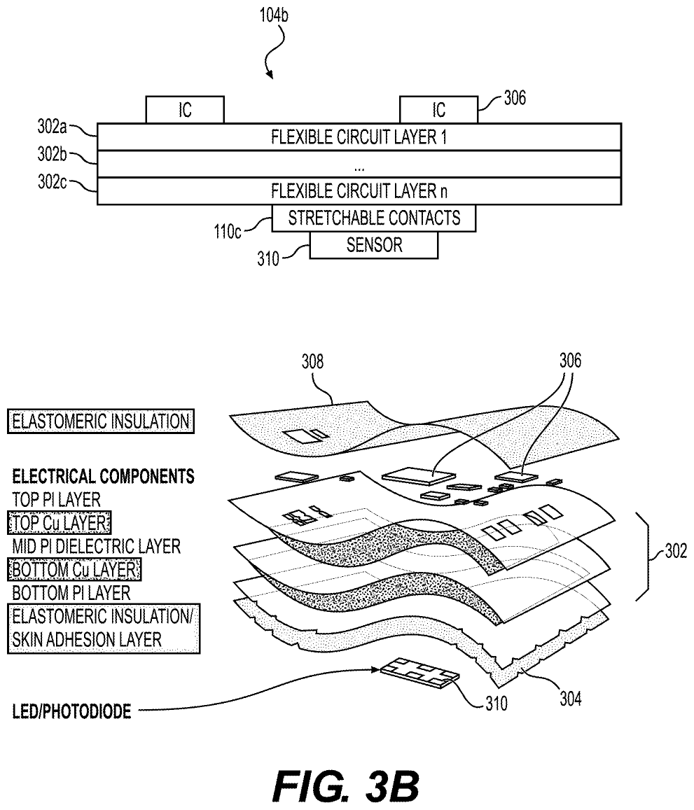

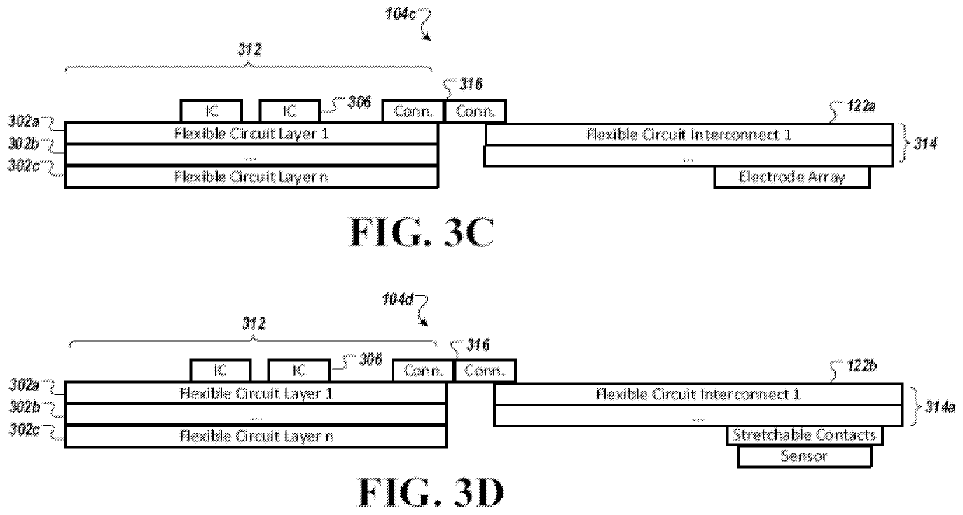

FIGS. 3A, 3B, 3C, and 3D each shows an example skin-wearable sensor system 104 (shown as 104a) of FIG. 1 in accordance with an illustrative embodiment.

In the example of FIG. 3A, the sensor system 104a includes multi-layer flexible circuits 302 (shown as 302a, 302b, 302c) that has an electrode array 110 (shown as 110b) fabricated on the underside of the bottom layer 302c. In some embodiments, the PI-Cu-PI-Cu-PI multilayers 302 may be stacked on a polydimethylsiloxane (PDMS)-coated 4-inch wafer (not shown—see FIG. 3). The fabricated circuit and electrodes may be retrieved from carrier substrates and transferred to a soft silicone elastomer 304. Functional microchips 306 (e.g., processor, analog-digital-converter, networking chipsets, among others) may be soldered on the exposed pads on the circuit and covered with the elastomer 308. A rechargeable Lithium battery (not shown) may be attached to the circuit, and the electrodes and the circuit may be linked with a flexible conductive film.

In the example of FIG. 3B, the sensor system 104b includes multi-layer flexible circuits 302 (shown as 302a, 302b, 302c) of FIG. 3A. The multi-layer flexible circuits 302 includes a stretchable pad 110 (shown as 110c) fabricated on the underside of the bottom layer 302c. The sensor system 104b further includes a LED and photodiode circuitries 310, or other integrated sensors described herein, that are attached to the stretchable pad 110c.

In the example of FIGS. 3C and 3D, the sensor system 104 (shown as 104c and 104d, respectively) includes a main circuit 312 comprising the multi-layer flexible circuits 302 (shown as 302a, 302b, 302c) of FIG. 3A. The main circuit 312 includes and is connected to an external sensor 122 (shown as 122a, 122b, respectively) via flexible circuits 314 through connectors 316. In the example shown in FIG. 3C, the external sensor 122a includes an electrode array (e.g., a stretchable electrode array). In the example shown in FIG. 3D, the external sensor 122b includes a stretchable pad 110 (shown as 110d) fabricated on the underside of the bottom layer of the flexible circuit membrane 314 (shown as 314a). The sensor system 104b further includes a LED and photodiode circuitries 310, or other integrated sensors described herein.

### Example Circuit

FIG. 4 shows an example circuit and layout of the sensor system (e.g., 104, 104a, 104b, 104c, 104d). In the example shown in FIG. 4, the sensor system includes printed stretchable electrodes fabricated on a soft elastomeric substrate. The circuit has a small dimension (6 cm²) and thickness (<2 mm) and is lightweight (1.63 g). After integrating a rechargeable battery (40 mAh capacity) with a slide switch, the total weight of the circuit becomes 3.17 g. The small battery allows the active wireless system to record multiple signals over 6 hours continuously.

The circuit design 400 includes integrated functional components, including a Bluetooth microprocessor that can deliver the measured sensor signals and motion signals to a mobile device. The received signal strength indication (RSSI) can provide a wireless communicating distance of 5 m with a maintained data transmission rate of 1104 bytes/s. The transmitted sensor signals and motion signals may be displayed and stored in a mobile device with a customized app. The soft, flexible circuit demonstrates mechanical reliability even under complete folding (180 degrees with 1.5 mm radius of curvature) during the cyclic loading (100 cycles).

In the example of FIG. 4, the circuit 400 include sensor ICs 402, antenna network 404, Bluetooth and microprocessor 406, voltage regulator 408, battery charging ICs 410, and motion sensor ICs 412. Specific components description are provided in table 414. Example values of the component include 0.1 μF (c1, c2, c10, c12, c14, c19, c22, c26), 0.1 nF (c20), 1 nF (c8, c9), 1 μF (c7, c18, c23), 10 uF (c3, c4, c5, c6, c11, c13), 1 pF (c27), 15 pF (c15), 4.7 μF (c16, c17, c21), 12 pF (c24, c25, c28, c29), 30kΩ (R1, R2), 1MΩ (R3, R4), 2kΩ (R5), 100kΩ (R6), 2.2 uH (L1), 2.7 nH (L2), and 3.9 nH (L3).

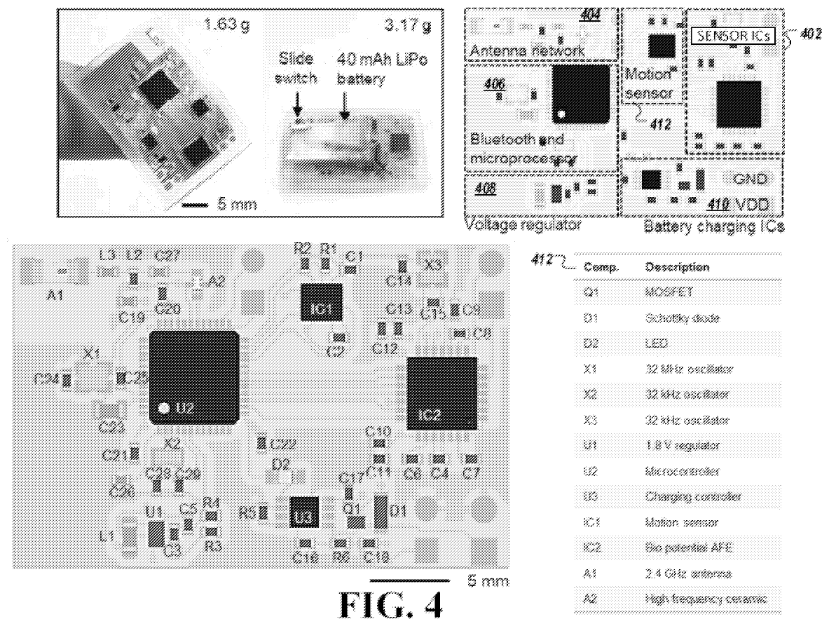

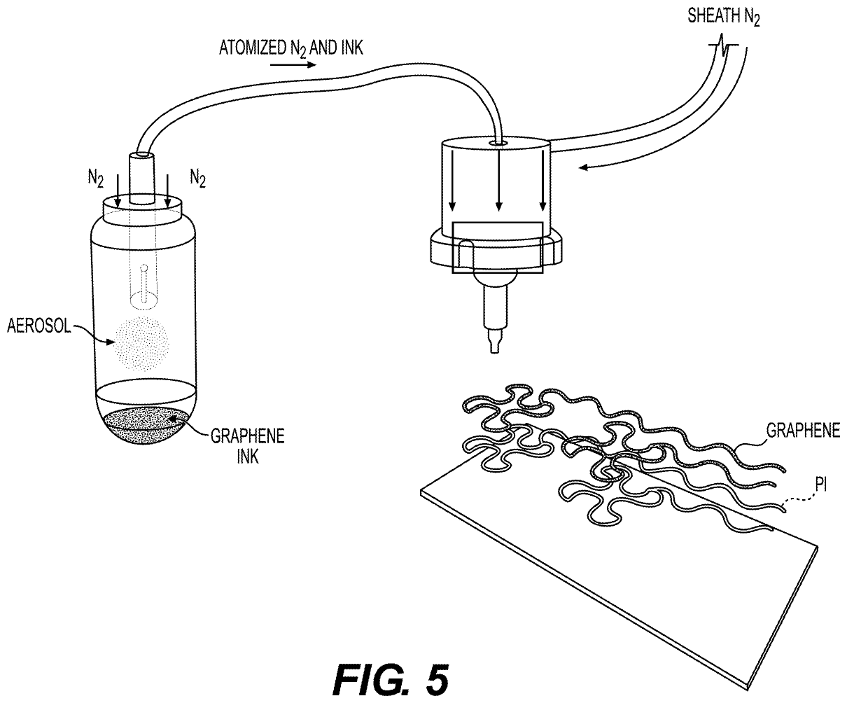

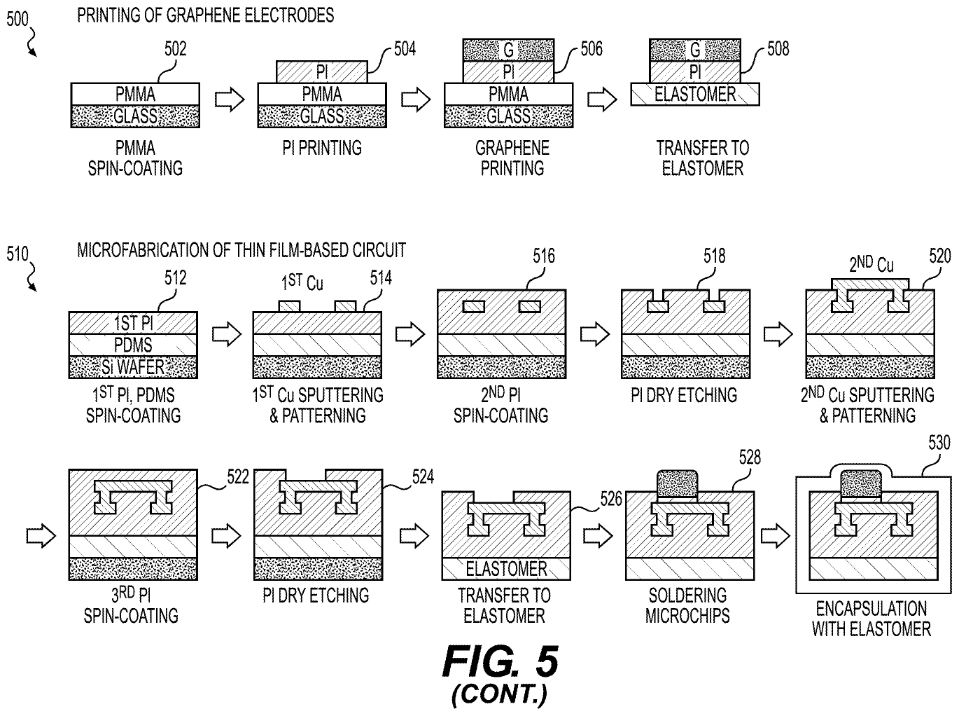

An example external sensor includes a skin-wearable electrodes fabricated using nanomanufacturing process. For example, the sensor may include two or more electrodes manufactured by aerosol jet printing (AJP) in a serpentine shape to provide stretchability. Conductive flexible films may be employed to connect between the sensor and the soft circuit. The width of the printed graphene membranes may be 0.55 mm. Printed graphene and PI membranes may be stacked on an elastomer, and graphene sheets may form a complete film without boundaries for enhanced conductivity. With the low-profile graphene membrane and PI layer inserted under the graphene, the electrodes can endure mechanical deformation during the fabrication and measurement process. FIG. 5 shows an example fabrication process for the sensor electrodes.

### Fabrication Details of a Wearable Electronics System

Graphene ink preparation. For the electrochemical exfoliation, 10 V may be applied between the graphite (Alfa Aesar) and Pt foil in an electrolyte solution of ammonium sulfate ((NH4)2SO4, Sigma-Aldrich). As-exfoliated graphene is purified using deionized water (DI water), the wet powder can be further filtered under vacuum to remove the residuals. The filtered wet powder of graphene can be dispersed in DI water and concentrated at 15%.

Table 1 shows example inks and printing parameters of PI and graphene. Resistivity and skin-contact impedance of the printed graphene may be around 2×10⁻³ Ωcm and 210.5 kΩ, respectively.

TABLE 1

| Factor | PI | Graphene |
|---|---|---|
| Solvent | N-Methyl-2- | N-Methyl-2- |
| | Pyrrolidone | Pyrrolidone |
| Concentration | 20% | 1% |
| Viscosity (cp) | 350 | 5 |
| Atomization mode | Pneumatic | Ultrasonic |
| Atomization rate (ccm) | 1000 (exhaust) | 40 |
| | 1100 (atomizing) | |
| Sheath rate (ccm) | 20 | 30 |
| Nozzle diameter (μm) | 300 | 200 |
| Stage temperature (° C.) | 80 | 70 |
| Sintering temperature (° C) | 250 | 200 |

### Graphene Electrode Printing

FIG. 5 shows a printing process 500 for graphene electrodes. In the example shown in FIG. 5, the process 500 includes spin-coating (502) PMMA (e.g., 950 PMMA, Kayaku Advanced Materials) on a glass at 1000 RPM for 30 s, bake at 200° C. for 2 min. The process 500 then includes atomizing (504) a polyimide (PI) ink (PI-2545, MicroSystems) dissolved in N-Methyl-2-Pyrrolidone (NMP, Sigma-Aldrich) in a 4:1 ratio in pneumatic atomizer of Aerosol jet printer, deposit using a 300-μm-diameter nozzle, and then cure at 250° C. for 1 h. The process 500 then includes printing (506) 1% of graphene ink dissolved in the NMP with a 200-μm-diameter nozzle. The process 500 then includes dissolving the printed electrodes in acetone. The process 500 then includes peeling (508) off the printed graphene layer with a water-soluble tape (ASWT-2, Aquasol) from the PMMA/slide glass and putting it on a silicone elastomer (1 mm thickness, 1:2 mixture of Ecoflex 00-30 and Gels, Smooth-On). Wash the tape with DI water.

### Circuit Fabrication.

FIG. 5 also shows a microfabrication process 510 for a thin film-based circuit. In the example shown in FIG. 5, process 510 includes spin-coating (512) PDMS (4:1 base-curing-agent ratio) on a Si wafer at 4000 RPM for 30 s. The 1st PI layer (PI-2610, MicroSystems) may be spin-coated at 2000 RPM for 60 s and then soft-baked at 100° C. for 5 min and hard-baked at 250° C. for 1 h. The process 510 then includes depositing (514) 0.5 μm thickness of Cu by sputtering.

The process 510 then includes spin-coating (516) photoresist (PR, Microposit SC1813, MicroChem) at 3000 RPM for 30 s. The work piece can then be aligned with a photomask and exposed to UV light, and developed with a developer.

The process 510 then includes etching (518) Cu with Cu etchant (APS-100, Transene. The 2nd PI layer (PI-2545) may be spin-coated at 2000 RPM for 60 s, and soft baked at 100° C. for 5 min. It may then be hard-baked at 240° C. for 1 h in a vacuum oven.

The process 510 then includes spin-coating (520) PR (AZ P4620, Integrated Micro Materials) at 2000 RPM for 30 sec, and soft-baked at 90° C. for 4 min. Photolithography may be performed to expose UV light with an intensity of 15 mJ/cm² for 100 s. The workpiece may then be developed with a developer (AZ-400K, Integrated Micro Materials) diluted with DI water (AZ-400K:DI water=1:4). Via hole may be etched with a reactive ion etcher (RIE). A 2nd Cu layer of 2 μm thickness may be deposited by sputtering. The process may then spin-coat the PR (AZ P4620) at 1500 RPM for 30 s, and soft bake at 90° C. for 4 min. Photolithography may be performed to expose UV light with intensity of 15 mJ/cm² for 120 s and develop. The process may etch exposed Cu with Cu etchant.

The process 510 then includes spin-coating (522) a 3rd PI layer (PI-2610) at 3000 RPM for 60 s and soft-baked it at 100° C. for 5 min and hard-baked at 240° C. for 1 h in a vacuum oven. The process may spin-coat PR (AZ P4620) at 900 RPM for 30 sec, and soft bakes at 90° C. for 4 min. Photolithography may be performed to expose UV light and develop the PR. The process may etch exposed the PI with RIE.

Process 510 then includes transferring (526) the circuit to an elastomer by peeling off the microfabricated circuit with a water-soluble tape from the PDMS/Si wafer. Process 510 then includes mounting (528) microchip components with screen-print low-temperature solder paste. Process 510 includes encapsulating the microchip components with an elastomer.

### EXAMPLES

An exemplary system and method are disclosed comprising a developed craniofacial volumetric muscle loss (VML) with live mouse models and a wireless nanomembrane non-invasive system that integrates skin-wearable printed sensors and electronics for real-time, continuous monitoring of VML. The craniofacial VML model, using biopsy punch-induced masseter muscle injury, shows impaired muscle regeneration and imbalanced muscle resident stem cell activities. To measure the electrophysiology of small and round masseter muscles of active mice during mastication, a wearable nanomembrane system is utilized that comprises stretchable graphene sensors that can be laminated to the skin over target muscles.

The thin-film electronics can be seamlessly mounted on the back of the mouse so as to allow its natural behavior while offering a long-range wireless recording of muscle activities. The noninvasive system provides highly sensitive electromyogram detection on masseter muscles with or without VML injury. Furthermore, it is demonstrated that the wireless sensor can monitor the recovery after transplantation surgery for craniofacial VML. It is shown that the functional recovery after surgeries is comparable between limb and masseter muscle transplantation to treat craniofacial VML. Overall, the presented comprehensive study of stem cell biology, nanomanufacturing, electrophysiology, and signal processing shows the enormous potential of our masseter muscle VML injury model and wearable electronic assay tool for both the mechanism study and the therapeutic development of craniofacial VML.

### Example 1: Real-Time Functional Assay of Craniofacial VML in Mouse Using Wireless Nanomembrane Electronics

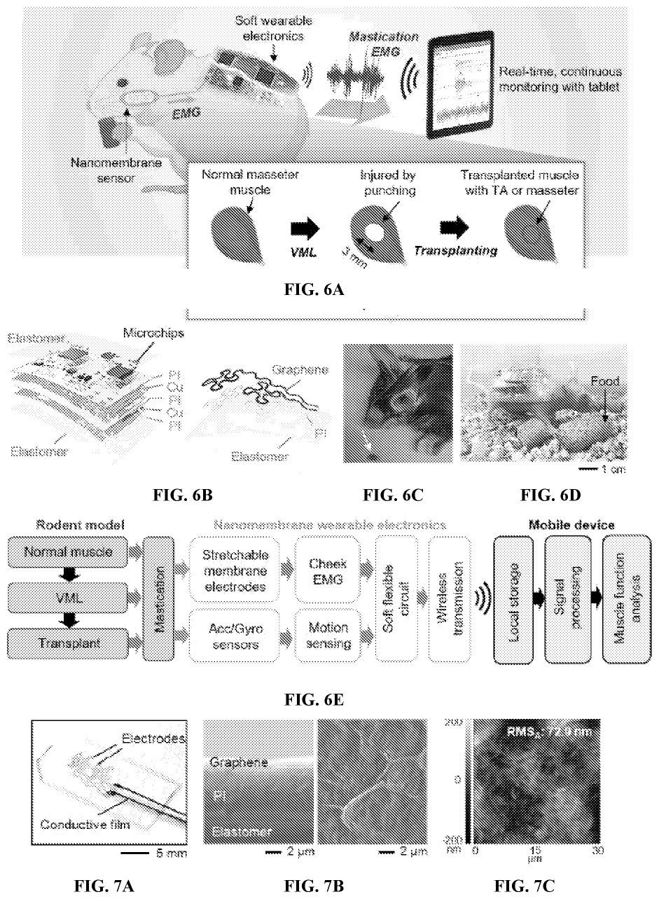

FIGS. 6A-6E show an overview of the real-time functional assay of craniofacial VML in mouse using wireless nanomembrane electronics.

Specifically, FIG. 6A shows a schematic illustration of a wireless electronic system on the skin of a mouse for a functional quantification of craniofacial VML of the masseter muscle. The mouse's EMG of the cheek area is continuously monitored on the mobile device. Inset shows the process of punch-induced VML of masseter and transplantation to treat VML of the masseter. Masseter muscle was injured by biopsy punching as a model of craniofacial VML. VML-injured masseter muscles are transplanted with a biopsied piece of tibialis anterior (TA) or masseter muscles to fill the injured area. This device includes non-invasive, stretchable electrodes to apply directly on the cheek skin of a mouse and a miniaturized, wireless, soft circuit to attach to the back of a mouse. During mastication, EMG data are measured by the electrodes on the mouse cheek skin and delivered to the microprocessor in the circuit for data processing. The signals are then wirelessly transmitted to an external mobile device for real-time, continuous data monitoring and saving. To establish VML on masseter muscles, a normal masseter muscle is injured by a 3-mm diameter biopsy punch. The injured muscle area is transplanted with 3-mm biopsied tibialis anterior (TA) or masseter muscle (inset of FIG. 6A).

FIG. 6B shows a schematic illustration of a multi-layered structure of a soft circuit (left) and stretchable sensor (right) on a soft elastomeric substrate, allowing for a conformal lamination on the mouse skin.

FIG. 6C shows a muscle biopsy to create VML in masseter muscles. The middle of the masseter muscles is biopsied by a 3 mm biopsy punch. Arrow indicates a muscle piece from a biopsy. FIG. 6D shows an optical image of an active mouse with the device during mastication. FIG. 6C displays the muscle biopsy used to produce masseter VML, while FIG. 6D shows that movement remains unhindered during monitoring.

FIG. 6E shows a flow chart capturing the quantitative metrics of analyzing muscle function. The flow chart shown in FIG. 6E denotes the quantitative metrics for wireless EMG measurement and analyzing muscle function. This wearable electronic system can monitor EMG activities in each of the three different craniofacial muscle conditions (normal, VML, treated VML by transplant) in our rodent model. Also, the motion sensor package can use a sensitive accelerometer and gyroscope, which identifies the mouse movements to distinguish mastication motions. A mobile device embeds a custom-designed app to offer real-time signal displaying and data storing to analyze muscle functions.

### Example 2 Characterization of Graphene Membrane Electrodes and Soft Wireless Circuits

To quantify the muscle function of our craniofacial VML model, EMG signals were monitored with a wearable wireless system, e.g., as described in relation to FIG. 4. Conventional EMG systems in animal studies [35, 38, 39] use bulky, invasive, needle-type-wired electrodes that penetrate into target muscle. The main issue with these systems are that they are unsuitable for use in small mice with active movements. In contrast, this study developed a non-invasive, miniaturized, soft electronic system to offer a wireless, high-fidelity recording of muscle functions with naturally moving mice.

The biocompatibility of a tissue-mounted sensor is a feature that may be used to provide safe and continuous use with adverse effects.[40, 41] In addition, cytotoxicity of the electrode can damage the skin cells of the VML-injured region when measuring muscle activities. Biocompatible characterizations for the printed graphene electrodes were conducted with human keratinocyte cells. The number of live cells on the graphene and control (polystyrene cell culture dish) was determined via fluorescence intensity.

Fluorescence images were taken of the cultured keratinocyte cells on two types of substrates, including a control (polystyrene petri dish, left) and graphene integrated on an elastomer (right). Tests were conducted using human primary keratinocyte cells cultured in an incubator at 37° C. with 5% CO₂. In the incubator, the material samples were placed in a 24-well plate, and 5000 keratinocytes/cm2 were seeded. After 7 days in the incubator, keratinocyte cells were washed with phosphate-buffered saline (Fisher Chemical) and dyed with 0.1 ml of calcein blue AM (Thermo Fisher) in 0.9 ml of the cultured medium. Keratinocytes and the reagent were additionally stored in the incubator of 37° C. for 10 min. The supernatant was then aliquoted in a 96-well plate for further biocompatibility.

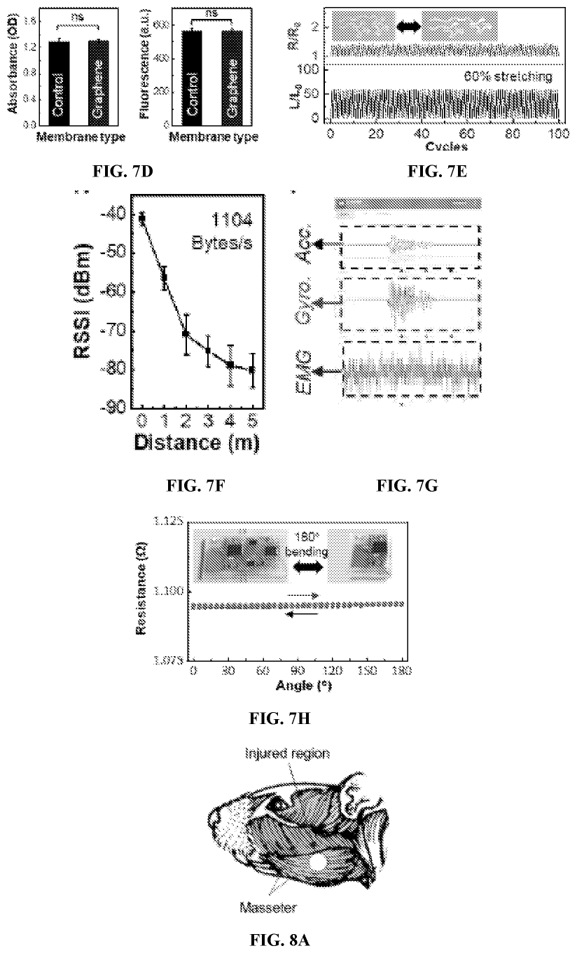

FIGS. 7A-7J show results of characterization of graphene membrane electrodes and wireless soft circuits. Specifically, FIG. 7A shows a photo of stretchable printed electrodes on an elastomeric membrane. FIG. 7B shows a cross-sectional (left) and top-view (right) SEM image of the multi-layered electrodes. FIG. 7C shows an AFM image indicating the surface roughness of the printed graphene. The average RMS is 72.9 nm, which captures the uniformity in the printed graphene layer. FIG. 7D shows a comparison of cell absorbance (left) and fluorescence (right) of cultured cells on the control and graphene. Data is analyzed 1-way ANOVA (ns=not statistically significant). Error bars represent standard error. FIG. 7E shows a graph representing relative resistance variation (top) according to the 60% of tensile strain change (bottom) for 100 cycles. It can be observed that the absorbance (left graph) and fluorescence (right graph) of cultured cells in FIG. 7D shows that the printed graphene electrodes have a negligible influence on cell viability. FIG. 7E represents the relative resistance variation of the electrodes (top) according to the 60% of tensile strain change (bottom) for 100 cycles, showing a reliable mechanical performance under strain.

FIG. 7F shows an RSSI response according to the Bluetooth communicating distance, showing maintained data transmission rate 1104 bytes/s. FIG. 7G shows a Mobile device application interface that displays real-time, continuous motion (accelerometer and gyroscope) and EMG signals from a wearable sensor system. FIG. 7H shows a resistance change of the flexible circuit upon cyclic loading (100 times with 180° bending at 1.5 mm radius of curvature), showing a negligible change of resistance.

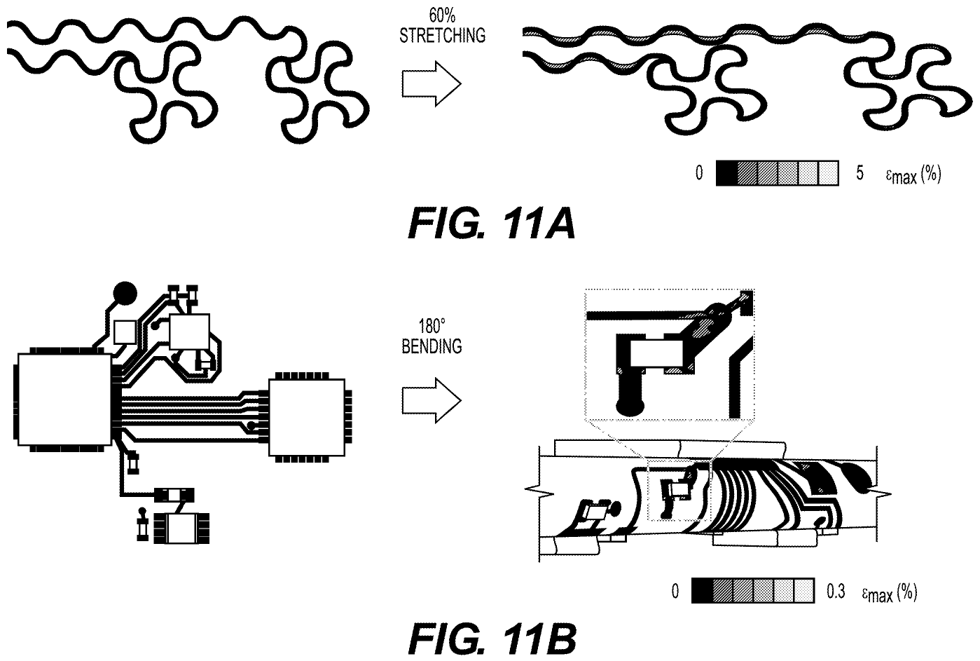

Analysis of received signal strength indication (RSSI) shows a successful wireless communicating distance of 5 m with a maintained data transmission rate of 1104 bytes/s (FIG. 7F). The transmitted EMG and motion signals are displayed and stored in a mobile device with a customized app (FIG. 7G). The soft, flexible circuit demonstrates mechanical reliability even under complete folding (180 degrees with 1.5 mm radius of curvature) during the cyclic loading (100 cycles; FIG. 7H), which agrees with the results from the computational modeling data (FIGS. 11A and 11B). FIGS. 11A-11B show results of computational mechanical simulations. Specifically, FIG. 11A shows a finite element analysis (FEA) results showing the mechanical compliance of printed electrodes before (left) and after 60% uniaxial stretching (right). FIG. 11B shows FEA results of the bendable circuit at 180°, showing no mechanical fracture.

### Example 3 Establishment of Craniofacial VML and Defective Masseter Muscle Regeneration

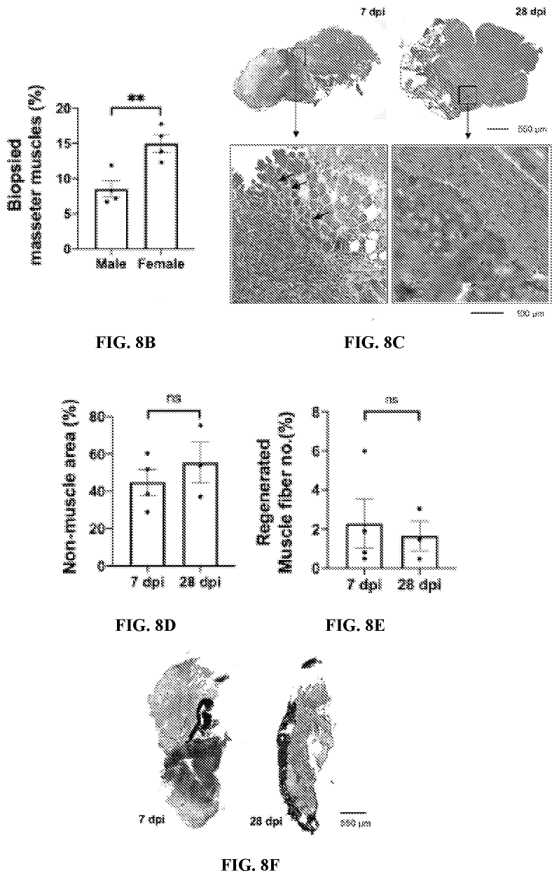

To establish craniofacial VML, masseter muscles were chosen as they are key muscles for mastication by pulling the mandible upward. Masseter muscles are composed of superficial and deep masseter muscles. The superficial masseter muscle is the thick and tendon-like portion and connects to the cheekbone, while the deep masseter muscle is smaller and connects to the mandible [42]. FIGS. 8A-8F show a study to establish craniofacial VML and defective masseter muscle regeneration.

Specifically, FIG. 8A shows an illustration of craniofacial VML of masseter muscle of a mouse. FIG. 8B shows a 3-mm biopsy punches induce a different degree of muscle loss between 6-month-old male and females. n=4 for each sex. Injury was applied to superficial masseter muscles using a 3 mm biopsy punch (FIG. 8A), which can generate about 8.5% and 15% loss of masseter muscle tissues by losing 8.5±2.3 gram and 7.9±1.2 gram of muscle masses from 6 months-old male and female mice, respectively (FIG. 8B). Masseter VML injury using a 3 mm biopsy on female mice presented a similar critical injury size (loss of 15%) volume compared to mouse limb VML injury,[16] which was not the case for male mice, although 3 mm biopsy on masseter muscles of male mice would be possible to induce functional impairment, female mice were used for the remainder of the experiments. To validate whether muscle regeneration occurs post-3 mm muscle biopsy induced injury in a craniofacial VML model, masseter muscles were sectioned at 7 days and 28 days of the post-VML injury.

FIG. 8C shows histology of masseter muscle after 7 days (left) and 28 days (right) post VML injury (dpi). Muscle sections were stained with Hematoxylin and Eosin to visualize nucleus (purple) and cytosol (red). Dotted area indicates non-muscle area. Small squared images are enlarged at the lower panels to show cellular elements of muscle tissues. Arrows in lower panel indicate muscle fiber-containing central nucleus, which is a feature of regenerating muscle fiber. FIG. 8D shows a non-muscle area occupies a majority of masseter muscles at 7 days and 28 days post-injury. Error bars represent the standard error of the mean (SEM). FIG. 8C shows histology data of a midpoint of the VML injury of masseter muscles colored through Hematoxylin and Eosin staining, which shows that non-muscle areas (dotted lines in FIG. 8C) are filled with non-muscle cells, maybe immune cells, and fibrosis. The non-muscle area occupied around 40% of injured masseter muscles at 7 days post-injury (dpi) and remained at 28 dpi (FIG. 8D). Limited muscle regeneration was observed at the rim of injured areas by measuring the cross-sectioned area of muscle fibers containing central nuclei, a feature of regenerated muscles (arrows in the bottom image of FIG. 8C).

FIG. 8E shows several regenerated muscle fibers were very low at 7 days and 28 days post-injury. Data is analyzed by Student t-test. ns=not significant statistically. FIG. 8F shows results of fibrosis of masseter muscle after 7 days (left) and 28 days (right) post VML injury. Muscle sections were stained with Massion's Trichrome staining to visualize fibrotic area (blue) from muscle tissues (brown). The regenerated muscle fiber number was about 2% of total muscle fibers in masseter muscles at 7 dpi and 28 dpi (FIG. 8E). Additionally, fibrosis in non-muscle areas of masseter muscles post-day 7 and day 28 VML injury was detected by Massion's Trichrome staining (FIG. 8F). Defective muscle regeneration after VML was supported by reduced satellite cells of masseter muscles with VML (FIG. 12), which are essential muscle stem cells for muscle regeneration upon injury [7] compared to ones of freeze-induced masseter muscle injury. FIGS. 12A-12D show results of filtered EMG signals of a post-VML-injured mouse. There is no signal variation for chewing behavior due to masseter muscle loss between FIG. 12A (non-eating) and FIG. 12B (eating). Chewing EMG signals of 3 mice with post-VML-injured after 30 days (FIG. 12C) and uninjured masseter muscle (FIG. 12D).

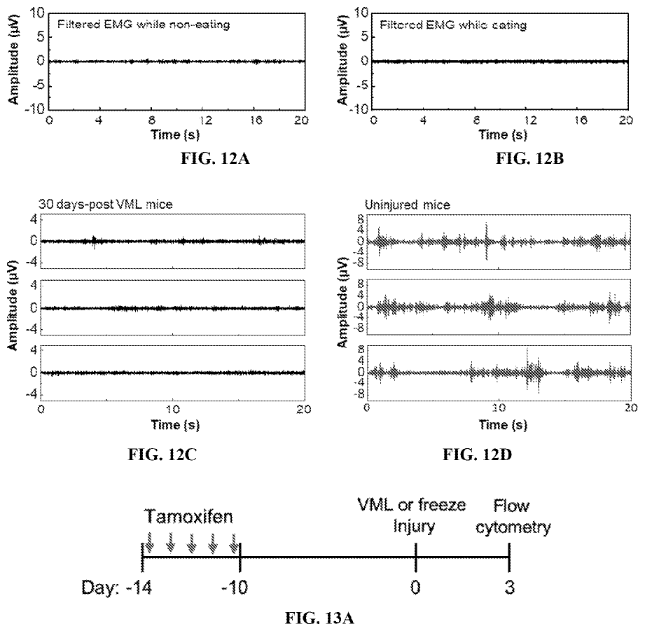

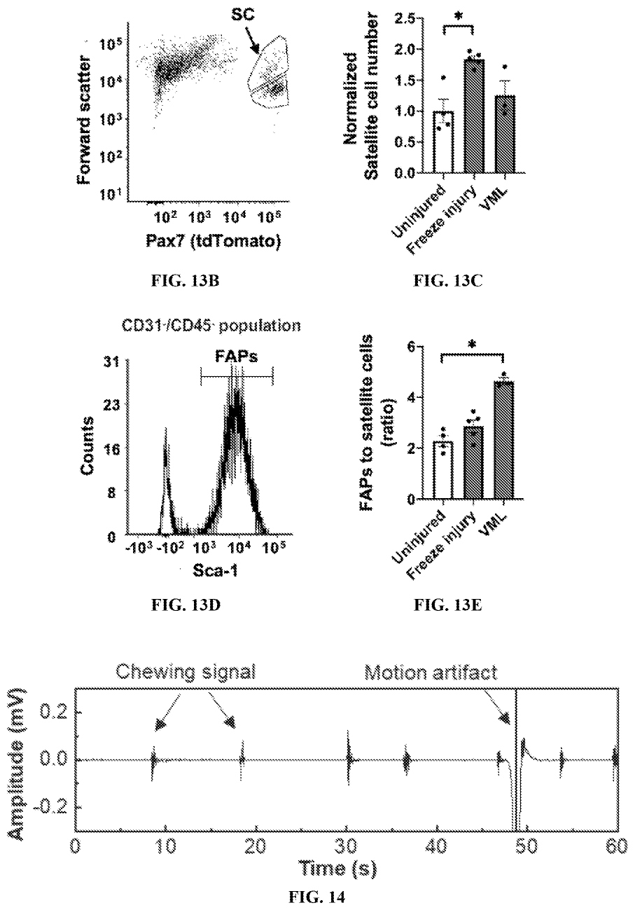

In addition, fibro adipose progenitor cells, a muscle mesenchymal stem cell that mediates fibrosis and fat deposit in chronic muscle injury [43, 44], were highly increased in VML masseter muscles than freeze-induced masseter muscle injury (FIG. 13), which may induce fibrosis in VML masseter muscles. These results prove that craniofacial VML generated by a 3-mm biopsy injury results in defective muscle regeneration similar to what is seen in limb VML experiments using the same method. [16]

FIGS. 13A-13E show results of dysregulated stem cells in masseter muscles at 3 days post-VML injury. Specifically, FIG. 13A shows a scheme of the experiments. Tamoxifen was injected Pax7^{cre/ERT};tdTomato mice for 5 days to induce tdTomato fluorescence expression in satellite cells. VML or freeze injury was performed 10 days after tamoxifen injection. Mononucleated cells were isolated for flow cytometry analysis at 3 days after injury. FIG. 13B shows a representative dot plot of satellite cells, which are gated by red fluorescent protein (tdTomato), using flow cytometry. FIG. 13C shows several satellite cells from VML-injured masseter muscles are comparable with one of uninjured masseter muscles. Satellite cell numbers are normalized to the average satellite cell number of uninjured muscles. Freeze-injured muscles are served as a positive control. Error bars represent the standard error of the mean (SEM). FIG. 13D shows a representative histogram of fibro adipose cells (FAPs), which is defined by surface markers (Cd31⁻, CD45⁻, Sca1⁺) using flow cytometry. FIG. 13E shows a ratio FAPs to satellite cells is increased in VML-injured masseter muscles. Data is analyzed 1-way ANOVA and Kruskal-Wallis method for post-hoc comparison. *p<0.05.

### Example 4 Demonstration of a Wireless, Wearable EMG System to Assess the Functionality of VML-Injured Masseter Muscles

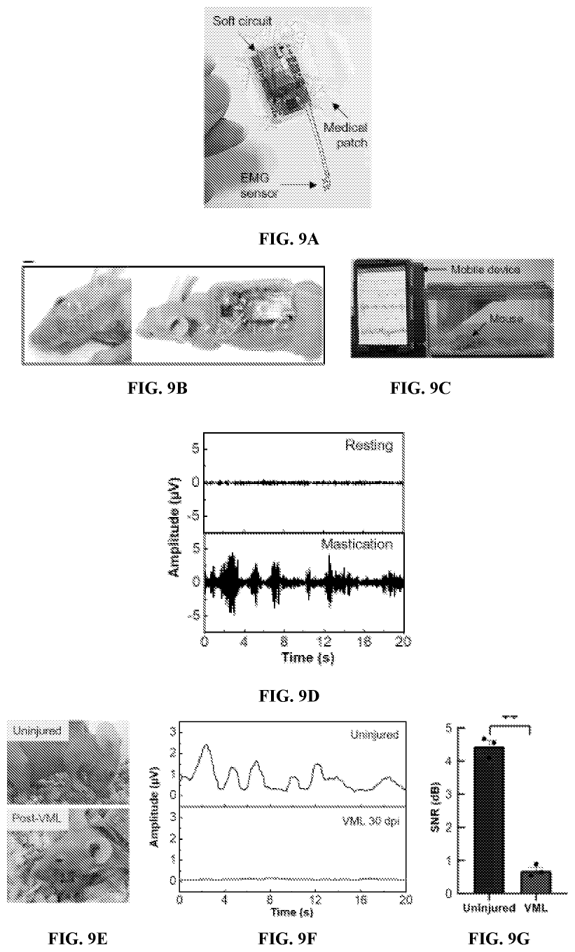

FIGS. 9A-9G show the operation of a wireless, wearable EMG system to assess the functionality of VML-injured masseter muscles. Specifically, FIG. 9A shows fully integrated wireless wearable electronics on a thin-film medical patch, e.g., for the non-invasive diagnosis of VML in mice.

FIG. 9B shows photos showing an ultrathin stretchable EMG sensor attached to the cheek (left) and a soft circuit on the back (right) of a nude mouse. The device is fully covered with a thin-film patch to easily mount on the skin of a target mouse. Stretchable EMG electrodes are directly attached to the mouse's cheek (left image in FIG. 9B), while the soft wireless circuit is mounted on the back of the body (right image in FIG. 9B).

FIG. 9C shows a photo of the experimental setup with a mouse in a cage. Real-time, continuous motion and EMG data are monitored and recorded by a mobile device with an embedded application. During the mouse's natural activities, including eating and roaming in the cage, real-time, continuous muscle functions are monitored by the wearable device and tablet.

FIG. 9D shows a comparison of real-time EMG signals, measured with an uninjured mouse, during the resting state (top) and mastication phase (bottom), which shows clear signal differences. FIG. 9D captures real-time EMG data recorded from a normal mouse during the resting state (top) and mastication (bottom), which displays an increased peak-to-peak voltage. The amplitude of mastication EMG signals is much smaller compared to typical intermittent motion artifacts measured by the mouse (FIG. 14). FIG. 14 shows filtered EMG signals of an uninjured mouse while eating. Motion artifacts make a significant and distinguishable amplitude compared with the chewing signals.

To quantify the function of post-VML-injured muscle, the EMG activity of masseter muscle was monitored at 30 days post-VML injury. FIG. 9E shows photos of an uninjured mouse (top) and a post-VML-injured mouse after 30 days (bottom). Arrow indicates the location of the VML-injured area. FIG. 9F shows representative RMS EMG signals during mastication corresponding to two cases in FIG. 9E. In addition, both root-mean-squared (RMS) EMG signals (FIG. 9F) and their signal-to-noise ratio (SNR) values (FIG. 9G) indicate a large significance (p-value<0.01) between the two groups, suggesting that the VML-injured muscle has not recovered a month after injury, which is consistent with our histology study results in FIG. 8. A clear signal difference is observed between the uninjured case (top) and VML-injured masseter muscles (bottom) at 30 days post-injury. FIG. 9G shows summarized EMG SNR data between the uninjured and post VML-injured masseter muscles during mastication. Data were analyzed by the unpaired two-tailed student t-test. **p<0.01.

### Example 5 Functional Recovery Monitoring of Post-Transplantation of VML-Injured Masseter Muscles

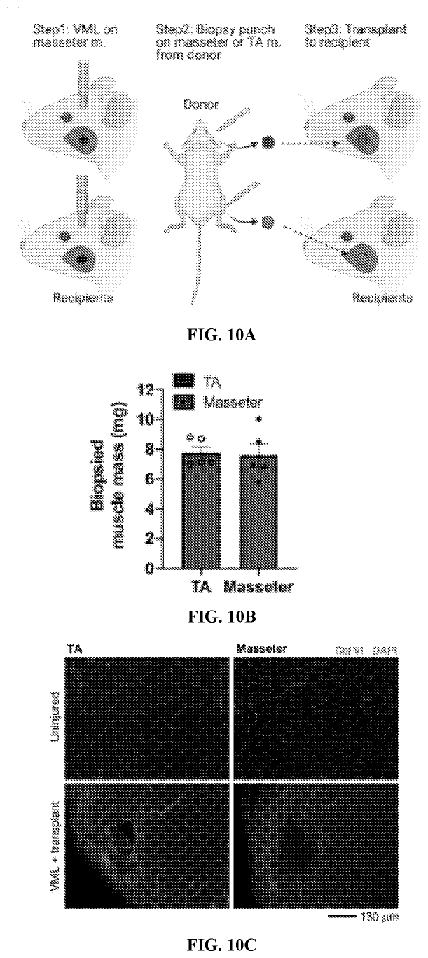

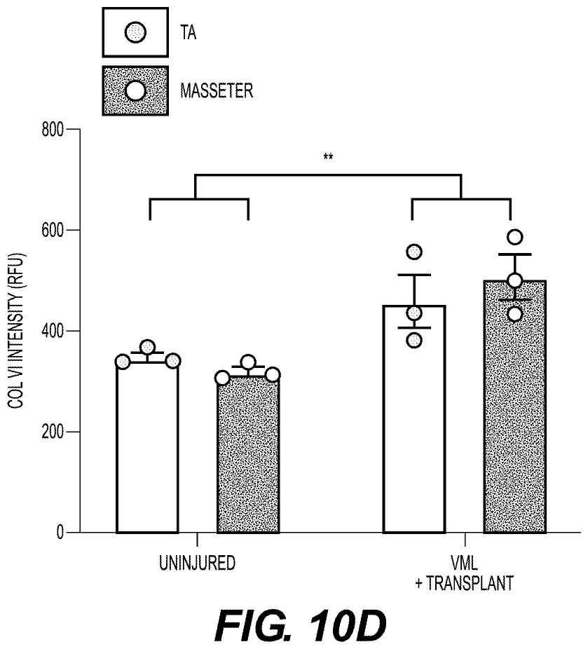

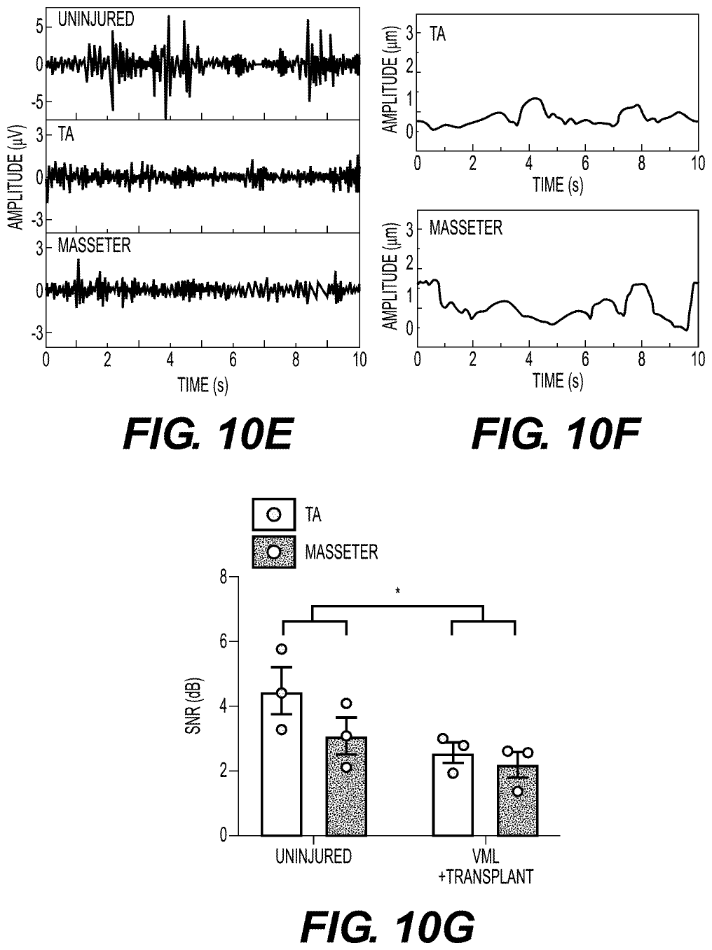

FIGS. 10A-10G show operation for monitoring the functional recovery of VML-injured masseter muscles post-transplantation. Specifically, FIG. 10A shows an experimental scheme showing biopsy-punch induced VML area of masseter muscles of immuno-deficient mice (NRG) filled with a biopsied piece from TA (blue) or masseter (red) muscles of wild-type mice. EMG measurement and fibrosis analysis are conducted at 30 days of post-VML injury/transplantation. Craniofacial VML was treated with autologous limb muscle transplantation in the clinic [45-47]. To evaluate the origins of transplanted muscles for craniofacial VML treatment, transplantation of masseter or tibialis anterior (TA) muscles were performed in the VML area of masseter muscles.

FIG. 10B shows uninjured, and VML-injured masseter muscles with TA and masseter muscle transplantation are sectioned and labeled with Col VI antibodies (red) to measure fibrosis. DAPI staining is used to label nuclei of muscle sections. Masseter or TA muscles were biopsied from wild-type mice using a 3-mm biopsy punch and transplanted into VML (3 mm biopsied area to ensure 1:1 volume match (FIG. 10B) of the masseter of severely immune-deficient mice.

FIG. 10C shows the averaged intensity of Col VI staining indicates that transplantation of TA or masseter muscle produced a comparable level of fibrosis in transplanted VML-injured masseter muscle; n=3 for each group and error bars represent the standard error of the mean. Data are analyzed with 2-way ANOVA. p**<0.01. These mice have been used in this transplant study due to the minimal likelihood of transplant rejection from the host's immune system.[48] Whole biopsied masseter or TA muscles were placed to align fibers between donors and recipients muscles to ensure proper muscle contractility. EMG activity of the transplanted masseter muscles was measured 30 days after surgery. To measure fibrosis, muscle sections from uninjured and VML-injured masseter muscles with TA and masseter muscle transplantation were labeled with anti-collagen VI (Col VI) antibodies (red) (FIG. 10C).

FIG. 10D shows comparison of filtered EMG of uninjured (top) and the VML-injured masseter muscles with TA (middle) or masseter (bottom) muscles transplant. The intensity of Col VI signals was measured to estimate the degree of fibrosis in uninjured (contralateral) and VML-injured masseter muscle with transplantation.

FIG. 10E shows RMS-EMG signals measured from TA (left) and masseter (right) muscle-transplanted masseter muscles. Fibrosis level is significantly higher in VML with transplanted masseter muscles compared to uninjured contralateral masseter muscles. However, transplantation of TA or masseter muscle does not produce a different level of fibrosis in transplanted VML-injured masseter muscle. Also, EMG activity is monitored with wearable membrane electronics to identify the functional recovery of masseter muscles after different muscle transplantation (FIG. 19). Decreased EMG responses were seen in both transplanted muscles compared to contralateral muscles during mastication (FIG. 10E).

FIG. 10F shows SNR values are decreased in VML with transplantation muscle group compared with uninjured muscles (n=3). TA or masseter muscle transplantation produces similar levels of functional recovery of VML-injured masseter muscles. The signal amplitude of both transplanted muscles appears comparable (FIG. 10F). The summarized SNR values in FIG. 10G show that muscle function in both VML/transplanted masseter muscles is partially recovered compared to the uninjured contralateral masseter muscles. Taken together, TA or masseter muscle transplantation produces similar levels of fibrosis and functional recovery of VML-injured masseter muscles.

### Experimental

Mice used in the study: C57BL/6J mice (Jax000664) (female n=6 and male n=4), Pax7^{CreERT2/CreERT2} mice (Jax017763), Rosa^{tdTomato/tdTomato} (tdTomato) (Jax007909), NU/J (Jax002019) (female n=7), NRG (NOD.Cg-Rag1^{tmlMom}Il2rg^{tm1Wjl}/SzJ; Jax007799) (female n=6) were purchased from Jackson Laboratories (Bar Harbor, ME; www.jax.org). Five to six months old mice were used, as noted in the figure legend. Homozygous Pax7^{CreERT2/CreERT2} male mice were crossed with homozygous Rosa^{tdTomato/tdTomato} (tdTomato) to obtain Pax7^{CreERT2/+}; Rosa^{tdTomato/+} (Pax7 Cre^{ERT2}-tdTomato) mice (female n=12). To label satellite cells with red fluorescence (tdTomato), tamoxifen, 1 mg (Sigma-Aldrich, St. Louis, MO) per 10 g body weight, was injected intraperitoneally once daily for 5 days. Experiments were performed in accordance with approved guidelines and ethical approval from Emory University's Institutional Animal Care and Use Committee and in compliance with the National Institutes of Health.

Muscle tissue injury and preparation for histology analysis: Mice were anesthetized by 2.5% isoflurane inhalation using a nose cone. For analgesia, mice were injected subcutaneously with 0.1 mg/kg buprenorphine SR (sustained release for 3 days) before muscle injury. The target injury area is the upper part of the superficial masseter muscle. For VML injury, masseter muscle was punched by 3-mm muscle biopsy punch, which was used to generate a critical size of VML in quadriceps muscles of mouse [16], by pushing biopsy punch down until it touched with a mandible bone. To avoid bleeding, the injury area was selected to avoid cutting the external carotid artery or posterior facial vein, both of which surround the upper and lower parts of deep and superficial masseter muscles, respectively. For freeze injury, a dry ice-cooled 4-mm metal probe was placed on the masseter muscles for 5 seconds, as described previously. [8] For transplantation surgery, the masseter muscles of NRG mice (recipient) were punched by a 3-mm muscle biopsy punch. Then, the biopsied area was filled with a biopsied piece of TA or masseter muscles from C57BL/6 mice (donor). Mass of biopsied pieces of TA and master muscles were equivalent. After injury or surgery, the skin was closed using an absorbable suture. Animals were euthanized by an overdose of isoflurane at the indicated time points. Superficial masseter muscle tissues were dissected and frozen in Tissue Freezing Medium (Triangle Biomedical Sciences) and stored at −80° C. Tissue cross-sections of 10 μm thickness were collected every 200 μm using a Leica CM1850 cryostat. To observe muscle histology, muscle sections were stained with hematoxylin and eosin (H&E) following the manufacture's instruction, imaged with Echo Revolve widefield microscope and analyzed using ImageJ. To detect fibrosis of muscle section, slides were stained using Massion's Trichrome Staining kit (Thermo Scientific) following the manufacturer's instruction (Advanced Microwave Staining Protocol). Slides were rehydrated in PBS before staining and imaged using Echo Revolve widefield microscope. To measure fibrosis in muscle tissues, muscle sections were immunostained with anti-collagen VI antibodies (Fitzgerald Industries International, 70R-CR009X, 1:300) and visualized AF594-conjugated donkey anti-rabbit antibodies. 4′,6-diamidino-2-phenylindole (DAPI) was used for nuclear staining.

Flow cytometry for cell analysis: To analyze the number of satellite cells and fibroadipose progenitor cells (FAPs) in injured muscles, muscles were dissected and digested with dispase II and collagenase II as previously described. [53] Isolated mononucleated cells were immunostained with the following antibodies: 1:400 CD45-PE (clone 30-F11; BD Biosciences), 1:4000 Sca-1-PE-Cy7 (clone D7, BD Biosciences), 1:400 CD31-PE (clone 390; eBiosciences). Fibroadipose progenitor cells were counted using the following criteria: CD31⁻/CD45⁻/Sca1⁺ and satellite cells are counted by tdTomato⁺ using BD LSR II cytometry analyzer and analyzed using FCS Expression 6 Flow software 6.01.

Fabrication of a nanomembrane electronic system: The integration of a soft platform with a microfabrication technique enabled all-in-one, wireless, and portable electronics. The device fabrication utilized multiple nanomanufacturing techniques, including a high-resolution printing process for graphene membrane electrodes [52, 54] and conventional photolithography, a metallization process for a thin-film-based circuit. [55, 56] For electrode fabrication, PI and graphene membranes were sequentially printed as a serpentine-patterned shape via AJP (Aerosol Jet 200, Optomec) on the polymethyl methacrylate (PMMA)-coated glass slide. For the circuit construction, PI-Cu-PI-Cu-PI multilayers were stacked on a polydimethylsiloxane (PDMS)-coated 4-inch wafer. The fabricated circuit and electrodes were retrieved from the carrier substrates and transferred to a soft silicone elastomer (1:1 mixture of Ecoflex 00-30 and Gels, Smooth-On). Functional microchips were soldered on the exposed Cu pads on the circuit and covered with the elastomer. A rechargeable LiPo battery (40 mAh, Adafruit) was integrated into the circuit. The electrodes and the circuit were linked with a flexible conductive film. A medical film (Tegaderm, 3M) was utilized to cover the device, which not only helped fix it onto a mouse's skin but also prevented external damage. An example description of the fabrication process is provided in relation to FIG. 5.

Mouse preparation to use wearable electronics: To acclimate mice to wearing the device, mice carried dummy circuits for 2-4 hours on their back a day before the experiment. To stimulate food consumption, food and water were removed for 18 hours before the experiment. On experiment day, mice were anesthetized with 2.5% isoflurane inhalation using a nose cone. If necessary, hair from the cheek and back area was removed with hair-removing lotion and wiped with alcohol pads to ensure alignment of membrane sensor or device with skin. Mice recovered on a heating pad after wearing the sensor and device. Three food pellets were provided when mice are active. The eating activity was recorded as a reference to EMG signals.

Signal processing and quantifying EMG signals: Masseter muscle EMG activity during mastication was selected for analysis. The EMG activity with a high motion signal was excluded from motion artifacts. Raw EMG signal was filtered by a second-order Butterworth band-pass filter at a cutoff frequency from 10 to 30 Hz. The filtered EMG was converted to the RMS signal to determine the peak amplitude and noise. The SNR was calculated as follows: [52, 57]

$${SNR}_{dB} = 10 {log}_{10} [ {( \frac{{A}_{signal}}{{A}_{noise}} )}^{2} ]$$

where A_{signal} is the amplitude of RMS EMG at chewing and Aₙₒᵢₛₑ is the amplitude during non-eating. The SNR was collected 5 times and averaged for analysis.

Statistical analyses: Statistical analysis was performed using Prism 8.0. Results are expressed as the means±SEM. Experiments were repeated at least three times unless a different number of repeats is stated in the legend. Statistical testing was performed using the unpaired t-test (Welch's t-test) if two groups were compared, 1-way ANOVA analysis and Kruskal-Wallis method for post-hoc comparison if more than two groups were compared, or 2-way ANOVA analysis if samples with 2 independent variables were compared, as stated in the figure legends. p<0.05 was considered statistically significant. The statistical method, p-values, and sample numbers are indicated in the figure legends. Power analysis of animal experiments was performed (Table 2).

TABLE 2
Power analysis of an animal study

| | | Effect size | | |
|---|---|---|---|---|
| Figure | P value | (d or *f) | Power | Analysis method |
| FIG. 3B | >0.01 | 2.157 | 0.89 | T-test, |
| | | | | two independent means |
| FIG. 3D | Not significantly | 1.527 | 0.44 | T-test, |
| | different | | | two independent means |
| FIG. 3E | Not significantly | 0.315 | 0.06 | T-test, |
| | different | | | two independent means |
| FIG. 4G | >0.01 | 12.523 | 1 | T-test, |
| | | | | two independent means |
| FIG. 5B | Not significantly | 0.105* | 0.05 | T-test, |
| | different | | | two independent means |
| FIG. 5D | >0.01 | 18.3* | 1 | F test, |
| | | | | ANCOVA: fixed effects, |
| | | | | main effects and |
| | | | | interactions |
| FIG. 5G | >0.05 | 7.141* | 1 | F test, |
| | | | | ANCOVA: fixed effects, |
| | | | | mainb effects and |
| | | | | interactions |
| FIG. S6C | >0.05 | 1.168* | 0.9 | F test |
| | | | | ANOVA: fixed effects, |
| | | | | omnibus, one-way |
| FIG. S6E | >0.05 | 3.319* | 1 | F test, |
| | | | | ANOVA: fixed effects, |
| | | | | omnibus, one-way |

### Discussion

The craniofacial region contains about 60 muscles that are vital for daily life functions, including eye movements, food uptake, respiration, and facial expressions.[1, 2]Although head and limb muscles are comparable to contractile organs, head muscles have several unique features compared to limb muscles, including distinctive embryonic origins [1, 3-5] and differential susceptibility to different types of muscular dystrophies.[6] Even though skeletal muscle is capable of regenerating damaged muscles via activation of muscle-specific stem cells, called satellite cells,[7] regeneration capacity varies between muscles. For example, masseter muscles that are critical for mastication have less regenerative capacity than tibialis anterior (TA) muscles[8] because masseter muscles contain fewer satellite cells that show delayed differentiation compared to satellite cells of limb muscles.[9] In contrast, satellite cells of other craniofacial muscles, such as extraocular muscles, show increased regenerative capacities relative to limb satellite cells.[10, 11] Therefore, understanding the unique features of specific muscles could lead to the development of targeted therapeutic approaches for the treatment of muscle injury.

Volumetric muscle loss (VML) refers to the traumatic or surgical loss of skeletal muscle tissues, which leads to chronic muscle weakness and impaired muscle function.[12]VML is often associated with military casualties as well as civilian vehicle accidents or gunshot injuries. VML is a clinically challenging problem since it requires surgical autologous muscle transplantation, which causes significant donor site morbidity.[13] Therefore, many research groups have been focused on muscle regeneration using myogenic cell therapies and extracellular matrix development in an animal extremity VML model.[14-17] Among the injury-caused VML, craniofacial injury with soft tissue penetration is a significant portion of battlefield injury[18] and civilian trauma injury. [19] Craniofacial VML causes loss of muscle function and severe cosmetic deformities, which may lead to social isolation and psychological depression.[19, 20] Several works have investigated VML of sheet-like muscles, which resemble the architecture of craniofacial muscles, using thin trunk muscles, including rat abdominal muscles [21-23] and rat latissimus dorsi. [20, 24] Studies have been conducted on VML on craniofacial muscles of large animals, such as zygomaticus muscles of sheep, emphasizing the pathophysiological differences between limb and craniofacial VML.[25] However, a craniofacial VML mouse model using actual craniofacial muscles has not been reported yet due to the small size of the craniofacial muscles of a mouse. A potential challenge in developing the craniofacial VML mouse model is the lack of functional assay tools that can monitor the regeneration and recovery of injured craniofacial muscles in the active mouse in a non-invasive manner. Current existing electromyogram (EMG) systems have limitations for longitudinal study using mouse models due to the bulky system, which requires invasive metal sensors, wires, and multiple electronic components.[26-28] Recent advances in wearable electronics have enabled wireless monitoring of various physiological signals that can be measured on the skin.[29-31] Compact device integration on a soft elastomeric platform can provide comfortable wearability without motion artifacts caused by cumbersome wires and rigid systems.[32, 33] The use of non-invasive and ergonomic factors in the monitoring system/device prevents restriction of movement during measurement, thus allowing us to monitor the physiological response in a natural ambulatory environment.

Here, this paper introduces nanomembrane electronics to measure real-time muscle electromyography on the skin of mouse masseter muscles with or without biopsy punch-induced VML. It was confirmed that the masseter VML model shows impaired muscle regeneration. To measure the function of VML-injured masseter muscles in active mice, a wireless and wearable electronic system was used to provide real-time EMG monitoring. This system includes ultrathin, low-profile, lightweight, and stretchable membrane sensors based on biocompatible graphene and thin-film soft circuits for data processing, which offers seamless mounting on the skin of mice without disrupting their natural behavior. In vivo demonstration of the EMG recording on the masseter muscles of mice validates the functionality of the wearable system that can clearly distinguish the signal difference between mice with and without craniofacial VML. Described is the use a wireless, non-invasive, soft EMG system in active and moving mice. Table 3 captures the work described herein compared to the prior reports in terms of electrode type, measurement type, recording system, target muscle, and data recording condition.[26, 27, 34-38] In addition, this system monitors the functional recovery after transplantation surgery to treat VML. It was shown that there is increased fibrosis and reduced EMG activities of VML-injured muscles following transplantation, regardless of the source of donor's muscles.

TABLE 3
Comparison of rodent model study with EMG measurements.

| | | Non- | | | | |
|---|---|---|---|---|---|---|
| | Electrode | invasive | Data | Target | Recording | |
| Reference | type | electrode | transmission | muscle | condition | Application |
| This work | Stretchable | Yes | Wireless | Masseter | Freely | Monitoring of |
| | graphene | | | muscles | moving and | craniofacial VML |
| | membrane | | | | mastication | and functional |
| | | | | | | recovery with |
| | | | | | | transplanted |
| | | | | | | muscles |
| K. | Flexible gold | No | Wire | Trapezius | During sleep | Identifying the |
| Kompotis | wires | | | muscles | | effect of rocking on |
| et al. | | | | | | sleep quality |
| (27) | | | | | | |
| D. P. | Rigid | No | Wire | Diaphragm | Anesthetized | Evaluating |
| Burns et | stainless- | | | and external | | diaphragm |
| al. | steel needle | | | intercostal | | dysfunction by |
| (36) | | | | muscles | | respiratory muscle |
| | | | | | | weakness |
| A. Silvani | Flexible | No | Wire | Neck and | During sleep | Monitoring of |
| et al. | stainless- | | | tibialis | | muscle activity |
| (37) | steel wire | | | anterior | | during sleep |
| | | | | muscles | | |
| Z. Ahmed | Rigid | No | Wire | External | Anesthetized | Monitoring of lower |
| (35) | stainless- | | | urinary | | urinary tract with |
| | steel needle | | | sphincter | | spinal cord injury |
| | | | | muscles | | |
| D S | Rigid | No | Wire | Facial muscle | Freely | Monitoring of |
| Freedman | stainless- | | | | moving | optogenetic |
| et al. | steel needle | | | | | behavior of mice |
| (26) | | | | | | |
| M. | Rigid | No | Wire | lower leg | Limited | Identifying motor |
| Hadizipasic | borosilicate | | | flexor and | moving | neuron loss of mice |
| et al. | glass | | | extensor | | with amyotrophic |
| (38) | | | | muscles | | lateral sclerosis |
| B. M. | Flexible | No | Wire | Tensor fascia | Anesthetized | Evaluating the |
| Sicari et | stainless- | | | latae muscles | | healing of VML by |
| al. | steel wire | | | | | extracellular matrix |
| (17) | | | | | | scaffold |

### Additional Discussion

Although the current study achieved meaningful continuous EMG monitoring in mice, motion artifacts affected signal analysis. Despite the low-profile and soft membrane electrodes, the device's size was slightly bigger than the target muscle. Also, it was observed that a testing mouse occasionally attempted to scratch the device, which can be resolved by further miniaturizing a circuit and sensor for the 2nd-generation device [49, 50]. Additionally, a motion sensor was introduced to exclude EMG signals at high motion activity to collect the EMG activity during mastication. An algorithm based on machine learning could offer automated signal discrimination and behavioral classification for further study [51, 52]. Since the EMG signal could be slightly different depending on the area to which the electrode was attached, constant localization with increasing the number of samples was demanded for accurate results. Nevertheless, the newly developed wireless EMG system demonstrated enough sensitivity to determine the function of masseter muscles. Transplant experiments were performed to verify the effectiveness of the craniofacial reconstitution surgery with autologous limb muscle graft [45-47] using EMG sensors. Although better outcomes (such as higher EMG) may be obtained if VML of masseter muscles were transplanted with masseter muscles due to recovery of original muscle type (type I for masseter) as well as resident stem cells, veins and nerves, the results are comparable when VML of masseter muscles were transplanted with limb muscles (TA, majority of muscle is type IIa/b). However, the fibrotic tissues around transplanted muscle tissues or cellulose tissues of the skin incision area would be huddles of accurate EMG measurements. This result again emphasized that precise sensor localization and increasing the number of samples are necessary for more accurate results. Overall, the device produced statistically different signals to distinguish between normal, the VML injured, and transplanted VML injured masseter muscles.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

The following patents, applications, and publications, as listed below and throughout this document are hereby incorporated by reference in their entirety herein.
[1]. D. M. Noden, P. Francis-West, The differentiation and morphogenesis of craniofacial muscles. Dev. Dyn. 235, 1194-1218 (2006).
[2]. F. Wachtler, M. Jacob, Origin and development of the cranial skeletal muscles. Bibl. Anat., 24-46 (1986).
[3]. P. Bailey, T. Holowacz, A. B. Lassar, The origin of skeletal muscle stem cells in the embryo and the adult. Curr. Opin. Cell Biol. 13, 679-689 (2001).
[4]. R. C. Mootoosamy, S. Dietrich, Distinct regulatory cascades for head and trunk myogenesis. Development 129, 573-583 (2002).
[5]. B. Christ, C. P. Ordahl, Early stages of chick somite development. Anat. Embryol. (Berl) 191, 381-396 (1995).
[6]. A. E. Emery, The muscular dystrophies. BMJ 317, 991-995 (1998).
[7]. C. Lepper, T. A. Partridge, C. M. Fan, An absolute requirement for Pax7-positive satellite cells in acute injury-induced skeletal muscle regeneration. Development 138, 3639-3646 (2011).
[8]. G. K. Pavlath, D. Thaloor, T. A. Rando, M. Cheong, A. W. English, B. Zheng, Heterogeneity among muscle precursor cells in adult skeletal muscles with differing regenerative capacities. Dev. Dyn. 212, 495-508 (1998).
[9]. Y. Ono, L. Boldrin, P. Knopp, J. E. Morgan, P. S. Zammit, Muscle satellite cells are a functionally heterogeneous population in both somite-derived and branchiomeric muscles. Dev. Biol. 337, 29-41 (2010).
[10]. L. K. McLoon, K. M. Thorstenson, A. Solomon, M. P. Lewis, Myogenic precursor cells in craniofacial muscles. Oral. Dis. 13, 134-140 (2007).
[11]. P. Stuelsatz, A. Shearer, Y. Li, L. A. Muir, N. Ieronimakis, Q. W. Shen, I. Kirillova, Z. Yablonka-Reuveni, Extraocular muscle satellite cells are high performance myo-engines retaining efficient regenerative capacity in dystrophin deficiency. Dev. Biol. 397, 31-44 (2015).
[12]. B. F. Grogan, J. R. Hsu, C. Skeletal Trauma Research, Volumetric muscle loss. J. Am. Acad. Orthop. Surg. 19 Suppl 1, S35-37 (2011).
[13]. C. H. Lin, Y. T. Lin, J. T. Yeh, C. T. Chen, Free functioning muscle transfer for lower extremity posttraumatic composite structure and functional defect. Plast. Reconstr. Surg. 119, 2118-2126 (2007).
[14]. X. Wu, B. T. Corona, X. Chen, T. J. Walters, A standardized rat model of volumetric muscle loss injury for the development of tissue engineering therapies. Biores. Open Access 1, 280-290 (2012).
[15]. K. Garg, C. L. Ward, B. J. Hurtgen, J. M. Wilken, D. J. Stinner, J. C. Wenke, J. G. Owens, B. T. Corona, Volumetric muscle loss: persistent functional deficits beyond frank loss of tissue. J. Orthop. Res. 33, 40-46 (2015).
[16]. S. E. Anderson, W. M. Han, V. Srinivasa, M. Mohiuddin, M. A. Ruehle, J. Y. Moon, E. Shin, C. L. San Emeterio, M. E. Ogle, E. A. Botchwey, N. J. Willett, Y. C. Jang, Determination of a Critical Size Threshold for Volumetric Muscle Loss in the Mouse Quadriceps. Tissue Eng. Part C Methods 25, 59-70 (2019).
[17]. B. M. Sicari, V. Agrawal, B. F. Siu, C. J. Medberry, C. L. Dearth, N. J. Turner, S. F. Badylak, A murine model of volumetric muscle loss and a regenerative medicine approach for tissue replacement. Tissue Eng. Part A 18, 1941-1948 (2012).
[18]. T. A. Lew, J. A. Walker, J. C. Wenke, L. H. Blackbourne, R. G. Hale, Characterization of craniomaxillofacial battle injuries sustained by United States service members in the current conflicts of Iraq and Afghanistan. J. Oral. Maxillofac. Surg. 68, 3-7 (2010).
[19]. R. Gassner, T. Tuli, O. Hachl, A. Rudisch, H. Ulmer, Cranio-maxillofacial trauma: a 10 year review of 9,543 cases with 21,067 injuries. J. Craniomaxillofac. Surg. 31, 51-61 (2003).
[20]. A. De Sousa, Psychological issues in oral and maxillofacial reconstructive surgery. Br. J. Oral Maxillofac. Surg. 46, 661-664 (2008).
[21]. M. T. Conconi, P. De Coppi, S. Bellini, G. Zara, M. Sabatti, M. Marzaro, G. F. Zanon, P. G. Gamba, P. P. Pamigotto, G. G. Nussdorfer, Homologous muscle acellular matrix seeded with autologous myoblasts as a tissue-engineering approach to abdominal wall-defect repair. Biomaterials 26, 2567-2574 (2005).
[22]. P. De Coppi, S. Bellini, M. T. Conconi, M. Sabatti, E. Simonato, P. G. Gamba, G. G. Nussdorfer, P. P. Pamigotto, Myoblast-acellular skeletal muscle matrix constructs guarantee a long-term repair of experimental full-thickness abdominal wall defects. Tissue Eng 12, 1929-1936 (2006).
[23]. C. L. Dearth, P. F. Slivka, S. A. Stewart, T. J. Keane, J. K. Tay, R. Londono, Q. Goh, F. X. Pizza, S. F. Badylak, Inhibition of COX1/2 alters the host response and reduces ECM scaffold mediated constructive tissue remodeling in a rodent model of skeletal muscle injury. Acta Biomater 31, 50-60 (2016).
[24]. X. K. Chen, T. J. Walters, Muscle-derived decellularised extracellular matrix improves functional recovery in a rat latissimus dorsi muscle defect model. J. Plast. Reconstr. Aesthet. Surg. 66, 1750-1758 (2013).
[25]. B. L. Rodriguez, E. E. Vega-Soto, C. S. Kennedy, M. H. Nguyen, P. S. Cedema, L. M. Larkin, A tissue engineering approach for repairing craniofacial volumetric muscle loss in a sheep following a 2, 4, and 6-month recovery. PLoS One 15, e0239152 (2020).
[26]. D. S. Freedman, J. B. Schroeder, G. I. Telian, Z. Zhang, S. Sunil, J. T. Ritt, OptoZIF Drive: a 3D printed implant and assembly tool package for neural recording and optical stimulation in freely moving mice. J. Neural Eng. 13, 066013 (2016).
[27]. K. Kompotis, J. Hubbard, Y. Emmenegger, A. Perrault, M. Mühlethaler, S. Schwartz, L. Bayer, P. Franken, Rocking promotes sleep in mice through rhythmic stimulation of the vestibular system. Curr. Biol. 29, 392-401. e394 (2019).
[28]. B. M. Sicari, J. P. Rubin, C. L. Dearth, M. T. Wolf, F. Ambrosio, M. Boninger, N. J. Turner, D. J. Weber, T. W. Simpson, A. Wyse, E. H. Brown, J. L. Dziki, L. E. Fisher, S. Brown, S. F. Badylak, An acellular biologic scaffold promotes skeletal muscle formation in mice and humans with volumetric muscle loss. Sci Transl Med 6, 234ra258 (2014).
[29]. R. Herbert, J.-W. Jeong, W.-H. Yeo, Soft Material-Enabled Electronics for Medicine, Healthcare, and Human-Machine Interfaces. Materials 13, (2020).
[30]. H. R. Lim, H. S. Kim, R. Qazi, Y. T. Kwon, J. W. Jeong, W. H. Yeo, Advanced soft materials, sensor integrations, and applications of wearable flexible hybrid electronics in healthcare, energy, and environment. Adv. Mater. 32, 1901924 (2020).
[31]. Y. Liu, M. Pharr, G. A. Salvatore, Lab-on-skin: a review of flexible and stretchable electronics for wearable health monitoring. ACS nano 11, 9614-9635 (2017).
[32]. H. Kim, Y. S. Kim, M. Mahmood, S. Kwon, N. Zavanelli, H. S. Kim, Y. S. Rim, F. Epps, W. H. Yeo, Fully Integrated, Stretchable, Wireless Skin-Conformal Bioelectronics for Continuous Stress Monitoring in Daily Life. Adv. Sci. 7, 2000810 (2020).
[33]. S. Kwon, Y.-T. Kwon, Y.-S. Kim, H.-R. Lim, M. Mahmood, W.-H. Yeo, Skin-conformal, soft material-enabled bioelectronic system with minimized motion artifacts for reliable health and performance monitoring of athletes. Biosens. Bioelectron. 151, 111981 (2020).
[34]. B. M. Sicari, J. P. Rubin, C. L. Dearth, M. T. Wolf, F. Ambrosio, M. Boninger, N. J. Turner, D. J. Weber, T. W. Simpson, A. Wyse, An acellular biologic scaffold promotes skeletal muscle formation in mice and humans with volumetric muscle loss. Sci. Transl. Med. 6, 234ra258-234ra258 (2014).
[35]. Z. Ahmed, Effects of cathodal trans-spinal direct current stimulation on lower urinary tract function in normal and spinal cord injury mice with overactive bladder. J. Neural Eng. 14, 056002 (2017).
[36]. D. P. Burns, K. H. Murphy, E. F. Lucking, K. D. O'Halloran, Inspiratory pressure-generating capacity is preserved during ventilatory and non-ventilatory behaviours in young dystrophic mdx mice despite profound diaphragm muscle weakness. J. Physiol. (Lond.) 597, 831-848 (2019).
[37]. A. Silvani, R. Ferri, V. Lo Martire, S. Bastianini, C. Berteotti, A. Salvadè, G. Plazzi, M. Zucconi, L. Ferini-Strambi, C. L. Bassetti, Muscle activity during sleep in human subjects, rats, and mice: towards translational models of REM sleep without atonia. Sleep 40, zsx029 (2017).
[38]. M. Hadzipasic, W. Ni, M. Nagy, N. Steenrod, M. J. McGinley, A. Kaushal, E. Thomas, D. A. McCormick, A. L. Horwich, Reduced high-frequency motor neuron firing, EMG fractionation, and gait variability in awake walking ALS mice. Proc. Natl. Acad. Sci. U.S.A 113, E7600-E7609 (2016).
[39]. C.-W. Wu, G. W. Randolph, I.-C. Lu, P.-Y. Chang, Y.-T. Chen, P.-C. Hun, Y.-C. Lin, G. Dionigi, F.-Y. Chiang, Intraoperative neural monitoring in thyroid surgery: lessons learned from animal studies. Gland Surg. 5, 473 (2016).
[40]. D. McShan, P. C. Ray, H. Yu, Molecular toxicity mechanism of nanosilver. J. Food Drug Anal. 22, 116-127 (2014).
[41]. S. Choi, S. I. Han, D. Jung, H. J. Hwang, C. Lim, S. Bae, O. K. Park, C. M. Tschabrunn, M. Lee, S. Y. Bae, Highly conductive, stretchable and biocompatible Ag—Au core-sheath nanowire composite for wearable and implantable bioelectronics. Nat. Nanotechnol. 13, 1048-1056 (2018).
[42]. P. G. Cox, N. Jeffery, Reviewing the morphology of the jaw-closing musculature in squirrels, rats, and guinea pigs with contrast-enhanced microCT. Anat. Rec. (Hoboken) 294, 915-928 (2011).
[43]. A. W. Joe, L. Yi, A. Natarajan, F. Le Grand, L. So, J. Wang, M. A. Rudnicki, F. M. Rossi, Muscle injury activates resident fibro/adipogenic progenitors that facilitate myogenesis. Nat. Cell. Biol. 12, 153-163 (2010).
[44]. A. Uezumi, S. Fukada, N. Yamamoto, M. Ikemoto-Uezumi, M. Nakatani, M. Morita, A. Yamaguchi, H. Yamada, I. Nishino, Y. Hamada, K. Tsuchida, Identification and characterization of PDGFRalpha+ mesenchymal progenitors in human skeletal muscle. Cell Death Dis. 5, e1186 (2014).
[45]. B. Del Frari, T. Schoeller, G. Wechselberger, Reconstruction of large head and neck deformities: experience with free gracilis muscle and myocutaneous flaps. Microsurgery 30, 192-198 (2010).
[46]. G. M. Huemer, T. Bauer, G. Wechselberger, T. Schoeller, Gracilis muscle flap for aesthetic reconstruction in the head and neck region. Microsurgery 25, 196-202 (2005).
[47]. B. Del Frari, T. Schoeller, G. Wechselberger, Free gracilis muscle flap for treatment of a large temporoparietal defect. J Plast Surg Hand Surg 46, 204-206 (2012).
[48]. T. Pearson, L. D. Shultz, D. Miller, M. King, J. Laning, W. Fodor, A. Cuthbert, L. Burzenski, B. Gott, B. Lyons, Non-obese diabetic-recombination activating gene-1 (NOD-Rag 1 null) interleukin (IL)-2 receptor common gamma chain (IL 2 rγnull) null mice: a radioresistant model for human lymphohaematopoietic engraftment. Clin. Exp. Immunol. 154, 270-284 (2008).
[49]. H. Wu, D. Kong, Z. Ruan, P.-C. Hsu, S. Wang, Z. Yu, T. J. Carney, L. Hu, S. Fan, Y. Cui, A transparent electrode based on a metal nanotrough network. Nat. Nanotechnol. 8, 421-425 (2013).
[50]. S. Lee, D. Sasaki, D. Kim, M. Mori, T. Yokota, H. Lee, S. Park, K. Fukuda, M. Sekino, K. Matsuura, Ultrasoft electronics to monitor dynamically pulsing cardiomyocytes. Nat. Nanotechnol. 14, 156-160 (2019).
[51]. Y. S. Kim, M. Mahmood, Y. Lee, N. K. Kim, S. Kwon, R. Herbert, D. Kim, H. C. Cho, W. H. Yeo, All-in-One, Wireless, Stretchable Hybrid Electronics for Smart, Connected, and Ambulatory Physiological Monitoring. Adv. Sci. 6, 1900939 (2019).
[52]. Y.-T. Kwon, H. Kim, M. Mahmood, Y.-S. Kim, C. Demolder, W.-H. Yeo, Printed, Wireless, Soft Bioelectronics and Deep Learning Algorithm for Smart Human-Machine Interfaces. ACS Appl. Mater. Interfaces 12, 49398-49406 (2020).
[53]. H. J. Choo, A. Cutler, F. Rother, M. Bader, G. K. Pavlath, Karyopherin Alpha 1 Regulates Satellite Cell Proliferation and Survival by Modulating Nuclear Import. Stem Cells 34, 2784-2797 (2016).
[54]. Y.-T. Kwon, Y.-S. Kim, S. Kwon, M. Mahmood, H.-R. Lim, S.-W. Park, S.-O. Kang, J. J. Choi, R. Herbert, Y. C. Jang, All-printed nanomembrane wireless bioelectronics using a biocompatible solderable graphene for multimodal human-machine interfaces. Nat. Commun. 11, 1-11 (2020).
[55]. H. Kim, Y.-S. Kim, M. Mahmood, S. Kwon, F. Epps, Y. S. Rim, W.-H. Yeo, Wireless, continuous monitoring of daily stress and management practice via soft bioelectronics. Biosens. Bioelectron. 173, 112764 (2020).
[56]. Y. T. Kwon, Y. Lee, G. K. Berkmen, H. R. Lim, H. A. Jinnah, W. H. Yeo, Soft Material-Enabled, Active Wireless, Thin-Film Bioelectronics for Quantitative Diagnostics of Cervical Dystonia. Adv. Mater. Technol. 4, 1900458 (2019).
[57]. Y.-T. Kwon, J. J. Norton, A. Cutrone, H.-R. Lim, S. Kwon, J. J. Choi, H. S. Kim, Y. C. Jang, J. R. Wolpaw, W.-H. Yeo, Breathable, large-area epidermal electronic systems for recording electromyographic activity during operant conditioning of H-reflex. Biosens. Bioelectron. 165, 112404 (2020).

1.-12. (canceled)

13. An animal model system, comprising:
an animal subject comprising a wearable device comprising a skin-wearable printed sensor over the animal subject's skin, wherein the animal subject was administered an agent of interest, was subjected to an injury, or any combination thereof;
the skin-wearable printed sensor comprises one or more stretchable graphene sensors configured as a sensor selected from the group consisting of electrical sensor, impedance sensor, infrared sensor, electrocardiogram (ECG) sensor, an electroencephalogram (EEC) sensor, an electromyogram (EMG) sensor, or any combination thereof, wherein the skin-wearable printed sensor is used to monitor an electrophysiological parameter of a subject.

14. The animal model of claim 13, wherein the injury comprises biopsy punch-induced masseter muscle injury, wherein the animal model is a craniofacial VML model.

15.-20. (canceled)

21. A method for monitoring an electrophysiological parameter of a subject, the method comprising:
acquiring signals from a wearable device comprising a skin-wearable printed sensor over a subject's skin; and
assessing disease progression on the subject, an injury on the subject, or any combination thereof using the acquired signals to provide real-time, continuous monitoring of the electrophysiological parameter of a subject.

22. The method of claim 21, wherein the skin-wearable printed sensor comprises one or more stretchable graphene sensors.

23. The method of claim 21, wherein the sensor is selected from electrical sensor, impedance sensor, infrared sensor, or any combination thereof, wherein the electrical sensor is selected from an electrocardiogram (ECG) sensor, an electroencephalogram (EEC) sensor, an electromyogram (EMG) sensor, or any combination thereof.

24.-31. (canceled)

32. A method of identifying therapeutic agent, the method comprising:
contacting a wearable device comprising a skin-wearable printed sensor with a subject's skin;
acquiring signals from the wearable device on the subject's skin;
administering an agent of interest to the subject;
acquiring signals from the wearable device on the subject's skin following administration of the agent of interest;
comparing the signals of the subject before and after administration of the agent of interest; and
analyze a result from the comparison step to assess a physiological parameter of the subject;
wherein the physiological parameter provides an indication that the agent of interest is a therapeutic agent.

33. The method of claim 32, wherein the therapeutic agent improves an injury on the subject.

34.-39. (canceled)

40. The system of claim 13, wherein the skin-wearable printed sensor comprises:
at least two electrodes;
a conductive flexible film; and
an elastomeric substrate.

41. The system of claim 40, wherein the electrodes comprise a graphene layer in contact with a polyimide (PI) layer.

42. The system of claim 40, wherein the polyimide layer is in contact with the elastomeric substrate.

43. The system of claim 40, wherein the conductive flexible film connects the device with electronics.

44. The system of claim 43, wherein the electronics comprise thin-film components.

45. The system of claim 43, wherein the electronics comprise wireless components.

46. The system of claim 44, wherein the thin film components comprise an antenna, bluetooth, microprocessor, acquisition electronics, battery, or any combination thereof.

47. The system of claim 13, wherein the skin-wearable printed sensor comprises one or more stretchable graphene sensors.

48. The system of claim 13, wherein the sensor is selected from electrical sensor, impedance sensor, infrared sensor, or any combination thereof.

49. The system of claim 48, wherein the electrical sensor is selected from an electrocardiogram (ECG) sensor, an electroencephalogram (EEC) sensor, an electromyogram (EMG) sensor, or any combination thereof.

50. The system of claim 13, wherein the skin-wearable printed sensor comprises a skin-wearable printed EMG sensor.

51.-64. (canceled)
